(19) United States
(12) Patent Application Publication
(10) Pub. No.: US 2023/0022045 A1
(43) Pub. Date: Jan. 26, 2023

(54) COMBINATION THERAPIES FOR THE TREATMENT OF CANCER

(71) Applicant: Board of Regents, The University of Texas System, Austin, TX (US)

(72) Inventors: David PIWNICA-WORMS, Houston, TX (US); Caleb GONZALEZ, Houston, TX (US); Seth GAMMON, Houston, TX (US)

(73) Assignee: Board of Regents, The University of Texas System, Austin, TX (US)

(21) Appl. No.: 17/782,147

(22) PCT Filed: Dec. 3, 2020
(86) PCT No.: PCT/US2020/063092

§ 371 Date: Jun. 2, 2022

Related U.S. Application Data
(60) Provisional application No. 62/942,987, filed on Dec. 3, 2019.

(51) Int. Cl. C07K 14/195; A61K 39/395; A61P 35/00; C07K 16/28

(52) U.S. Cl. CPC C07K 14/195; A61K 39/39541; A61P 35/00; C07K 16/2818; A61K 2039/505

(57) ABSTRACT

Combination therapies for the treatment of cancers are provided. In some embodiments, a flagellin derivative such as CBLB502 is administered in combination with an immune checkpoint therapy (e.g., an anti-PD 1 antibody and an anti-CTLA4 antibody) to treat a cancer in a mammalian subject. In some embodiments, the combination therapy is administered intratumorally or peritumorally.

This application claims the benefit of U.S. Provisional Patent Application No. 62/942,987, filed Dec. 3, 2019, the entirety of which is incorporated herein by reference.

### BACKGROUND OF THE INVENTION

### 1. Field of the Invention

The present invention relates generally to the field of molecular biology and medicine. More particularly, it concerns combination therapies for the treatment of cancer.

### 2. Description of Related Art

Despite the success of immune checkpoint therapies (ICT) in achieving durable responses in some cancer patients, not all patients respond to ICT therapies. Primary and adaptive resistance to immunotherapies present a considerable obstacle in achieving enhanced survival in a broader population of patients. Innate immune activators have been actively pursued for their antitumor potential, but cancer remains a significant clinical challenge. Clearly, there is a need for improved therapies for the treatment of cancer.

### SUMMARY OF THE INVENTION

The present disclosure, in some aspects, overcomes limitations in the prior art by providing new methods for the treatment of cancer. In some aspects, it has been shown that administration of a flagellin or derivatives (e.g., CBLB502) in combination with an immune checkpoint therapy (ICT) can be used to synergistically treat a cancer or treat cancers that are refractory to ICT alone, such as highly refractory triple negative breast cancer. In some embodiments, the ICT comprises or consists of inhibitors of CTLA-4 and PD-1, or inhibitors of CTLA-4 and PD-L1, such as inhibitory antibodies. In some embodiments, CBLB502 is administered to treat the cancer in combination with an anti-PD1 antibody and/or an anti-PD-L1 antibody, and the therapy may optionally further comprise administering an anti-CTLA-1 antibody. In some embodiments, the ICT and flagellin (e.g., CBLB502) are administered intratumorally or peritumorally.

As shown in the below examples, a syngeneic 4T1 mammary carcinoma murine model for established highly refractory triple negative breast cancer showed enhanced survival when treated intratumorally with either the Toll-like receptor 5 (TLR5) agonist flagellin or CBLB502, a flagellin derivative, in combination with intra-peritoneal antibodies targeting CTLA-4 and PD-1. Peritumoral administration may be particularly useful for the treatment of one or more nonpalpable tumors in a subject or patient. Long-term survivor mice showed immunologic memory upon tumor re-challenge and a distinctive immune activating cytokine profile that engaged both innate and adaptive components of the immune system. Low serum levels of G-CSF correlated with enhanced survival. These results illustrate that an ICT in combination with innate immune activation with TLR5 agonists (e.g., CBLB502) can be used to treat ICT-refractory solid tumors. Additional data showed that CBLB502 can improve the efficacy of not only anti-PD1, but also with an anti-PD-L1 therapy, optionally further in combination with anti-CTLA4 therapy.

An aspect of the present invention relates to a method of treating a cancer in a mammalian subject, comprising administering to the subject a therapeutically effective amount of: (i) a TLR5 agonist; and (ii) an immune checkpoint therapy (ICT). In some embodiments, the TLR5 agonist is flagellin or a flagellin derivative. In some embodiments, the TLR5 agonist is flagellin, CBLB502, or a CBLB502 derivative (e.g., a truncated or shortened version of CBLB502 that retains the ability to function as an agonist of TLR5). In some embodiments, the CBLB502 derivative has at least 95% sequence identity to CBLB502 and retains the ability to function as an agonist of TLR5. In some embodiments, the CBLB502 or CBLB502 derivative can be generated using a codon optimized sequence. The immune checkpoint therapy may comprise an anti-PD1 antibody, an anti-PD-L1 antibody, an anti-CTLA4 antibody; an anti-LAG3 antibody, an anti-TIM-3 antibody, an anti-VISTA antibody, an anti-TIGIT antibody, an anti-KIR antibody, an anti-CD47 antibody, an anti-B7-H3 antibody, an anti-B7-H4 antibody, an ICOS agonist, an OX40 agonist, and/or an IDO inhibitor. In some embodiments, the immune checkpoint therapy comprises: (a) an anti-PD1 antibody or an anti-PD-L1 antibody; and (b) an anti-CTLA4 antibody. In some embodiments, the anti-PD1 antibody is Pembrolizumab, Nivolumab, REGN2810, BMS-936558, SHR1210, IBI308, PDR001, BGB-A317, BCD-100, or JS001. In some embodiments, the anti-PDL1 antibody is Avelumab, Atezolizumab, Durvalumab, KN035, MPDL3280A, MEDI4736, or BMS-936559. In some embodiments, the anti-CTLA4 antibody is Ipilimumab or Tremelimumab. In some embodiments, the CBLB502 is administered to the subject. In some embodiments, about 5 μg/mL to 150 μg/mL of CBLB502 is administered to the subject. The administration may be intratumoral, peritumoral, intravenous, parenteral, subcutaneous, or intrathecal. In some embodiments, the administration is intratumoral or peritumoral. In some embodiments, CBLB502, an anti-PD1 antibody, and an anti-CTLA4 antibody are administered to the subject. The administration may be intratumoral, peritumoral, intravenous, parenteral, subcutaneous, or intrathecal. In some embodiments, the administration is intratumoral or peritumoral. In some embodiments, the cancer is an ICT-refractory cancer or an ICT-refractory solid tumor. The cancer may be a melanoma, a breast cancer, a lung cancer, a prostate cancer, a pancreatic cancer, a head and neck cancer, a liver cancer, an ovarian cancer, a nonpalpable cancer, or a lymphoma. In some embodiments, the cancer is a melanoma or a breast cancer (e.g., a triple negative breast cancer). The subject may be a human, dog, cat, horse, or cow. In some embodiments, the subject is a human.

Another aspect of the present invention relates to a pharmaceutical composition comprising CBLB502, and an immune checkpoint therapy (ICT), wherein the pharmaceutical composition is formulated for injection, intratumoral administration, or peritumoral administration. In some embodiments, the immune checkpoint therapy is an anti-PD1 antibody, an anti-PD-L1 antibody, or an anti-CTLA4 antibody. The pharmaceutical composition may comprise both an anti-PD-L1 antibody and an anti-CTLA4 antibody. The pharmaceutical composition may comprises an anti-PD1 antibody, an anti-PD1 antibody, and an anti-CTLA4 antibody. The pharmaceutical composition may comprise both an anti-PD1 antibody and an anti-CTLA4 antibody. The anti-PD1 antibody may be Pembrolizumab, Nivolumab, REGN2810, BMS-936558, SHR1210, IBI308, PDR001, BGB-A317, BCD-100, or JS001. The anti-PDL1 antibody may be Avelumab, Atezolizumab, Durvalumab, KN035, MPDL3280A, MEDI4736, or BMS-936559. In some embodiments, the anti-CTLA4 antibody is Ipilimumab or Tremelimumab.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawings(s) will be provided by the Office upon request and payment of the necessary fee.

FIGS. 1A-F: In vitro characterization of 4T1 murine mammary carcinoma cells response to incubation with TLR5 agonists. 4T1 cells stably expressing pκB₅:IκBαFLuc were stimulated with the indicated ligand at t=0 and bioluminescence activity imaged every 5 minutes for 4 hours. Data are displayed as normalized photon flux values (average fold-initial, fold-vehicle). (FIG. 1A) 4T1 cells stably expressing pκB₅:IκBαFLuc were treated with increasing flagellin concentrations: 1 ng/ml (n=7), 10 ng/ml (n=2), 50 ng/ml (n=2), 100 ng/ml (n=7), 500 ng/ml (n=7), 750 ng/ml (n=2), 1 μg/ml (n=7), 2.5 μg/ml (n=7), 3 μg/ml (n=5), 4 μg/ml (n=5), 5 μg/ml (n=7), 7.5 μg/ml (n=5), 10 μg/ml (n=7), and TNFα 20 ng/ml (n=7). Error bars represent S.E.M. for the indicated number of independent experiments. (FIG. 1B) The half maximal effective concentration (EC50) of flagellin in 4T1 cells is >10⁴ ng/mL in this model. (FIG. 1C) 4T1 cells stably expressing pκB₅:IκBαFLuc were treated with increasing CBLB502 concentrations: 0.1 ng/ml (n=3), 0.5 ng/ml (n=3), 1 ng/ml (n=3) 1.5 ng/ml (n=3), 2.5 ng/ml (n=3), 5 ng/ml (n=3), 10 ng/ml (n=3), 25 ng/ml (n=3), 100 ng/ml (n=3), 1 μg/ml (n=3), and TNFα 20 ng/ml (n=3). (FIG. 1D) The half maximal effective concentration (EC50) of CBLB502 in 4T1 cells is approximately 3.1 ng/mL in this model. Error bars represent S.E.M. for the indicated number of independent experiments. (FIG. 1E) In vitro cytokine profile of 62 cytokines secreted from 4T1 FUGW cells in response to overnight treatment with vehicle control (PBS) or CBLB502 (1 μg/mL). Data are displayed as normalized density, average values of three membranes per treatment. Error bars represent standard-error of three membranes. (FIG. 1F) Data from FIG. 1E displayed as the fold difference between vehicle control (PBS) and CBLB502 (1 μg/mL) treatments. Data are shown as normalized density, mean values of three membranes per treatment (fold-initial, fold-vehicle).

FIG. 2: Combination treatment with flagellin and ICT enhance survival. Kaplan-Meier survival analysis of BALB/c mice implanted with orthotopic 4T1 FUGW-FL florescent and bioluminescent reporter cells at week 0 and treated with flagellin with or without ICT from week 2 to week 4. Mice treated with vehicle control (PBS) (n=25), ICT (n=23), flagellin (n=22) and flagellin+ICT treatment (n=22) were compared. Treatment with flagellin+ICT treatment had a statistically significant effect on survival (p=0.01, Log-rank test; p=0.02, Gehan-Breslow-Wilcoxon test) compared to treatment with vehicle control. ICT only and flagellin only treatments did not show detectable differences (p=0.1, Log-rank test; p=0.1 Gehan-Breslow-Wilcoxon test and p=0.2 and Log-rank test; p=0.2 Gehan-Breslow-Wilcoxon test, respectively).

FIGS. 3A-D: Flagellin murine experiments. BALB/c mice implanted with orthotopic 4T1 FUGW-FL tumor cells. (FIG. 3A) Tumor size of vehicle-treated negative control mice (n=25) measured by bioluminescence imaging (total photon flux, left panel) and caliper measurements (tumor volume, right panel). All vehicles-treated mice died by week 8. (FIG. 3B) Tumor size of ICT-treated mice (n=23) measured by bioluminescence imaging (total photon flux, left panel) and caliper measurements (tumor volume, right panel). All ICT-treated mice died by week 8. (FIG. 3C) Tumor size of flagellin-treated mice (n=22) measured by bioluminescence imaging (total photon flux, left panel) and caliper measurements (tumor volume, right panel). One flagellin-treated mouse, FIG. 5A—Mouse 13, was tumor-free for 40 weeks before it was re-challenge with 4T1 FUGW-FL tumor (Table 4). (FIG. 3D) Tumor size of flagellin plus ICT-treated mice (n=22) measured by bioluminescence imaging (total photon flux, left panel) and caliper measurements (tumor volume, right panel). Mice 2, 6, and 7 were tumor-free for 54, 51, and 51 weeks, respectively, before they were re-challenge with 4T1 FUGW-FL tumor cells (Table 4).

FIG. 4: Combination treatment with CBLB502 (low dose) and ICT enhance survival. Kaplan-Meier survival analysis of BALB/c mice implanted with orthotopic 4T1 FUGW-FL florescent and bioluminescent reporter tumor cells at week 0 and treated with CBLB502 (low dose) with or without ICT from week 2 to week 4. Mice treated with vehicle control (PBS) (n=27), ICT (n=25), CBLB502 (low dose) (n=30) and CBLB502 (low dose)+ICT (n=30) were compared. Treatment CBLB502 (low dose)+ICT had a statistically detectable effect on survival (p=0.001, Log-rank test; p=0.001, Gehan-Breslow-Wilcoxon test) compared to treatment with vehicle control. ICT alone treatment did not show a detectable difference by Log-rank test (p=0.1), but did show a detectable difference with the Gehan-Breslow-Wilcoxon test (p=0.04). CBLB502 (low dose) alone treatment did not show detectable difference (p=0.8, Log-rank test; p=0.3 Gehan-Breslow-Wilcoxon test).

FIGS. 5A-D: CBLB502 low dose murine experiments. BALB/c mice implanted with orthotopic 4T1 FUGW-FL tumor cells. (FIG. 5A) Tumor size of vehicle-treated negative control mice (n=27) measured by bioluminescence imaging (total photon flux, left panel) and caliper measurements (tumor volume, right panel). One vehicle control mouse, FIG. 5A—Mouse 25, was tumor-free for 18 weeks before it was re-challenge with 4T1 FUGW-FL tumor (Table 4). (FIG. 5B) Tumor size of ICT-treated mice (n=25) measured by bioluminescence imaging (total photon flux, left panel) and caliper measurements (tumor volume, right panel). One ICT-treated mouse, FIG. 5B—Mouse 23, was tumor-free for 22 weeks before it was re-challenge with 4T1 FUGW-FL tumor (Table 4). (FIG. 5C) Tumor size of CBLB502-treated mice (n=30) measured by bioluminescence imaging (total photon flux, left panel) and caliper measurements (tumor volume, right panel). All CBLB502-treated mice died by week 9. (FIG. 5D) Tumor size of CBLB502 plus ICT-treated mice (n=30) measured by bioluminescence imaging (total photon flux, left panel) and caliper measurements (tumor volume, right panel). Mice 1, 4, 10, 19, 22 and 26 were tumor-free for 40, 40, 38, 22, 18 and 18 weeks, respectively, before they were re-challenge with 4T1 FUGW-FL tumor cells (Table 4).

FIGS. 6A-B: Re-challenge experiment. (FIG. 6A) Kaplan-Meier survival analysis of BALB/c mice re-challenged with orthotopic 4T1 FUGW-FL tumor cells in the contralateral (left) fourth mammary fat pad and age-matched control tumor-naïve mice. All mice were injected at week 0 with 4T1 FUGW-FL tumor cells and tumors were allowed to grow without therapeutic intervention. Re-challenged mice showed 80% survival rate (p=0.0001, Log-rank test; p=0.0001 Gehan-Breslow-Wilcoxon). (FIG. 6B) Tumor size of tumor-naïve mice (n=14) measured by bioluminescence imaging (total photon flux, left panel) and caliper measurements (tumor volume, right panel). All tumor-naïve, tumor challenged-mice died by week 6. (FIG. 6C) Tumor size of re-challenged mice (n=16) measured by bioluminescence imaging (total photon flux, left panel) and caliper measurements (tumor volume, right panel). Only three tumor survivor mice died due to tumor burden: FIG. 10C—Mouse 5 (CBLB502 high dose), FIG. 5B—Mouse 23 (ICT), and FIG. 5D—Mouse 1 (CBLB502 low dose (i.t.) Table 4). All other tumor survivor mice were tumor-free for at least 60 weeks post orthotopic tumor re-challenge.

FIGS. 7A-B: Peripheral blood cytokine profile. (FIG. 7A) Profile of 32 peripheral blood-borne cytokines of tumor-free mice and mice challenged with 4T1 FUGW-FL tumor: tumor-free mice (healthy mice); tumor-bearing mice, vehicle (PBS) control (non-survivors); tumor-bearing mice, treatment failure (non-survivors); and tumor-bearing mice, treatment responders (survivors) during weeks 5 to 7. G-CSF is marked in red to highlight the difference in levels among the different groups. (FIG. 7B) Profile of 32 peripheral blood-borne cytokines of mice re-challenged with 4T1 FUGW-FL tumor: tumor naïve, tumor-bearing mice (non-survivors), tumor survivor, re-challenge failure (non-survivors) and tumor survivor, re-challenge survivor (long-term survivors) three weeks post orthotopic tumor implantation. G-CSF is marked in red to highlight the difference in expression among the different groups.

FIG. 8: Combination treatment with CBLB502 (i.t.) and ICT (i.p.) enhance survival. Kaplan-Meier survival analysis of C57BL/6J mice implanted with B16-F10 cells at Day 0 and treated with CBLB502 with or without ICT three days post tumor implantation. Mice treated with vehicle control (PBS) (n=20, red), ICT (n=15, green), CBLB502 (n=14, orange) and CBLB502+ICT treatment (n=39, blue) were compared. Treatment with CBLB502+ICT treatment had a statistically significant effect on survival (p=0.001, Log-rank test; p=0.003, Gehan-Breslow-Wilcoxon test) compared to treatment with vehicle control.

FIG. 9: Higher CBLB502 dose combined with ICT treatment does not enhance survival. Kaplan-Meier survival analysis of BALB/c mice implanted with orthotopic 4T1 cells stably transfected with EF1a FUGW florescent and bioluminescent reporter (4T1 FUGW) at week 0 and treated with CBLB502 high dose with or without ICT from week 2 to week 4. Mice treated with vehicle control (PBS) (n=15), ICT (n=15), CBLB502 (n=30) and CBLB502 in combination with ICT (n=30) were compared. Treatment with a combination of CBLB502 and ICT did not result in long-term survivors, but had a statistical detectable effect on survival (p=0.0002, Log-rank test; p=0.0001, Gehan-Breslow-Wilcoxon test). Compared with vehicle control combination treatment shifted the median survival by one week. ICT alone did not show detectable difference (p=0.1, Log-rank test; p=0.1, Gehan-Breslow-Wilcoxon test). CBLB502 alone treatment did show detectable difference (p=0.01, Log-rank test; p=0.01 Gehan-Breslow-Wilcoxon test).

FIGS. 10A-D: CBLB502 high dose murine experiments. BALB/c mice implanted with orthotopic 4T1 FUGW-FL tumor cells. (FIG. 10A) Tumor size of vehicle-treated negative control mice (n=15) by bioluminescence imaging (total photon flux, left panel) and caliper measurements (tumor volume, right panel). All vehicle control mice were dead by week 7. (FIG. 10B) Tumor size of ICT treated mice (n=15) measured by bioluminescence imaging (total photon flux, left panel) and caliper measurements (tumor volume, right panel). All ICT control mice were dead by week 8. (FIG. 10C) Tumor size of CBLB502 high dose treated mice (n=15) measured by bioluminescence imaging (total photon flux, left panel) and caliper measurements (tumor volume, right panel). One CBLB502 high dose-treated mice was tumor-free for 51 weeks post orthotopic tumor implantation, before it was re-challenge with 4T1 FUGW-FL tumor (Table 4). (FIG. 10D) Tumor size of CBLB502 high dose in ICT treated mice (n=15) by bioluminescence imaging (total photon flux, left panel) and caliper measurements (tumor volume, right panel). All CBLB502 high dose treated with ICT died by week ten. However, two mice showed delayed tumor growth: mouse 7 and 10.

FIG. 11: Systemic administration of CBLB502. Kaplan-Meier survival analysis of BALB/c mice implanted with orthotopic 4T1 FUGW cells at week 0 and treated with CBLB502 administered through intraperitoneal injection (i.p.) with or without ICT treatment from week 2 to week 4. Mice treated with vehicle control (PBS) (n=7), ICT (n=6), CBLB502 (n=20) and CBLB502 i.p in combination with ICT (n=20) were compared. Treatment with a combination of CBLB502 i.p. with ICT had a statistically significant effect on survival (p=0.01, Log-rank test; p=0.01, Gehan-Breslow-Wilcoxon test) compared to treatment with vehicle control. ICT only and CBLB502 i.p. only treatments did not show detectable difference (p=0.1, Log-rank test; p=0.1 Gehan-Breslow-Wilcoxon test and p=0.4 and Log-rank test; p=0.3 Gehan-Breslow-Wilcoxon test, respectively).

FIGS. 12A-D: Systemic CBLB502 murine experiments. BALB/c mice implanted with orthotopic 4T1 FUGW tumor cells. (FIG. 12A) Tumor size of vehicle-treated negative control mice (n=7) by bioluminescence imaging (total photon flux, left panel) and caliper measurements (tumor volume, right panel). All vehicle control mice were dead by week 8. (FIG. 12B) Tumor size of ICT treated mice (n=6) measured by bioluminescence imaging (total photon flux, left panel) and caliper measurements (tumor volume, right panel). One ICT mouse was tumor-free for 18 weeks post orthotopic tumor implantation, before it was re-challenge with 4T1 FUGW-FL tumor (Table 4). (FIG. 12C) Tumor size of CBLB502 i.p. treated mice (n=20) measured by bioluminescence imaging (total photon flux, left panel) and caliper measurements (tumor volume, right panel). All CBLB502 i.p. control mice were dead by week 9. (FIG. 12D) Tumor size of CBLB502 i.p. with ICT treated mice (n=20) measured by bioluminescence imaging (total photon flux, left panel) and caliper measurements (tumor volume, right panel). Two CBLB502 i.p. treated with ICT mice were tumor-free for 22 weeks post orthotopic tumor implantation, before they were re-challenged with 4T1 FUGW-FL tumor (Table 4).

FIGS. 13A-D: B16-F10 melanoma tumor growth. C57BL/6J mice implanted with B16-F10 cells at Day 0 and treated with CBLB502 alone or with or without ICT three days post tumor implantation. (FIG. 13A) Tumor size of vehicle-treated negative control mice (n=20) assessed by caliper measurements (tumor volume). One vehicle control mouse, mouse 18, was tumor-free for 12 weeks. (FIG. 13B) Tumor size of ICT-treated mice (n=15) assessed by caliper measurements (tumor volume). All CBLB502-treated mice died by week 12. (FIG. 13C) Tumor size of CBLB502-treated mice (n=14) assessed by caliper measurements (tumor volume). One CBLB502 treated mouse, mouse 14, was tumor-free for 12 weeks. (FIG. 13D) Tumor size of CBLB502 plus ICT-treated mice (n=39) assessed by caliper measurements (tumor volume). Mice 3, 4, 6, 8, 9, and 10 were tumor-free for at least 77 weeks; mice 11 and 13 were tumor-free for at least 58 weeks; mice 33, 34, 35 and 38 were tumor-free for at least 12 weeks.

FIG. 14: Experimental design and timing of administration.

FIG. 15: Survival curve results.

### DESCRIPTION OF ILLUSTRATIVE EMBODIMENTS

The present invention, in some aspects, provides combination therapies for the treatment of cancers. In some aspects, a flagellin (e.g., CBLB502) is administered in combination with an ICT (e.g., antibody inhibitors of CTLA-4, PD-L1, and/or PD-1) to treat a cancer (e.g., a triple negative breast cancer). As shown in the below examples, these combinations may result in the synergistic treatment of cancers that do not respond to the ICT alone. In some embodiments, the ICT (e.g., antibody inhibitors of CTLA-4, PD-L1, and/or PD-1) and flagellin (e.g., CBLB502) are administered intratumorally.

### I. Flagellin and Flagellin Derivatives

Recent evidence indicates that therapies that harness innate immunity show promising antitumor potential (Goldberg and Sondel, 2015). In a number of studies, Salmonella typhimurium, a flagellated facultative intracellular bacteria, induce tumor regression in pre-clinical models (Frahm et al., 2015; Pawelek et al., 2003; Pawelek et al., 1997; Yu et al., 2012; Flentie et al., 2012; Ganai et al., 2009; Forbes, 2010; al-Ramadi et al., 2009). Building on this concept, therapeutic trials with Salmonella species are underway (multiple myeloma trial of orally administered Salmonella based Survivin vaccine. ClinicalTrials.gov Identifier: NCT03762291). Without wishing to be bound by any theory, the therapeutic effects of Salmonella are likely driven by bacteria antigenicity and activation of host immunity-mediated recognition of pathogen-associated molecular patterns by Toll-like receptors (TLRs) (Flentie et al., 2012; Kawasaki and Kawai, 2014; Rakoff-Nahoum and Medzhitov, 2009; Zheng et al., 2017). Many TLRs agonist have been shown to elicit antitumor activity (Garaude et al., 2012; Lu, 2014; Nguyen et al., 2013; Simone et al., 2009). In particular, treatment with bacterial flagellin, a TLR5 agonist (Hayashi et al., 2001), results in potent antitumor responses in various xenograft models for colon, breast, and prostate cancer as well as a number of mouse spontaneous tumor models (Rhee et al., 2008; Cai et al., 2011; Galli et al., 2010; Sfondrini et al., 2006). Interestingly, higher TLR5 expression levels correlate with enhanced survival in breast, lung, and ovarian cancer patients (Flentie et al., 2018). Although the precise mechanisms of TLR5-mediated antitumor effects remain to be elucidated, it is known that TLR5 mediates innate immune responses against bacterial flagellin (Hayashi et al., 2001), likely through activation of pro-inflammatory pathways, including NF-κB (Rhee et al., 2008; Flentie et al., 2018; Menendez et al., 2011). Thus, it is possible that the antitumor responses are a collateral effect of host immune response to flagellin. Bacterial flagellin has been viewed as a virulence factor that can contribute adhesion and invasion of host cells, but this protein may also function as an immune activator (Hajam et al., 2017). TLR5-mediated immunogenic response has led to the exploration of flagellin-derived reagents suitable for clinical application.

A. CBLB502

CBLB502 (Entolimod), is a recombinant flagellin protein fragment derived from Salmonella enterica that can act as a TLR5 agonist and can activate the NF-κB inflammatory response (Burdelya et al., 2008; Zhou et al., 2012). In pre-clinical studies, treatment with CBLB502 showed antitumor and anti-metastatic effects through activation of components of the innate immune system (Leigh et al., 2014; Hossain et al., 2014; Brackett et al., 2016; Yang et al., 2016; Burdelya et al., 2013). Safe systemic administration of CBLB502 has been demonstrated in rodents, non-human primates, and humans (Burdelya et al., 2013; ClinicalTrials.gov Identifier: NCT01527136). As shown in the below examples, in some embodiments, CBLB502 administered in combination with an immune checkpoint therapy (ICT) can be used to treat a cancer and may synergistically interact. The 4T1 breast cancer solid tumor model, a highly aggressive cancer refractory to standard therapies (Lechner et al., 2013; Song et al., 2018), was used in the studies in the examples and provides in vivo evidence that such combinations may be particularly useful for the treatment of cancers that are refractory to other therapies. In some embodiments, CBLB502 and the ICT are administered intratumorally.

CBLB502 (also referred to as Entolimod) is a toll-like receptor 5 (TLR5) agonist derived from Salmonella flagellin. CBLB502 has displayed some anti-inflammatory effects towards gut mucosal tissues (Xu et al., 2016). CBLB502 is further described in U.S. Pat. No. 10,265,390 and U.S. Pub. No. 2012/0208871, which are incorporated by reference herein in their entirety.

A variety of dosages of CBLB502 may be administered to a subject. It is anticipated that the therapeutically effective dosage of CBLB502 to produce an anti-cancer effect may be significantly reduced when administered in combination with an ICT. For example, CBLB502 can be administered to a subject, such as a human patient, in combination with an ICT to treat a cancer in the subject, wherein the CBLB502 is in a range of from about 0.001 mg/kg to about 200 mg/kg per day, from about 1 mg/kg to about 100 mg/kg per day, or about 1-50 mg/kg. The dosage may be at any dosage such as about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 25 mg/kg, 50 mg/kg, 75 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1 g/kg. In some preferred embodiments, the CBLB502 is administered intratumorally or peritumorally.

The therapeutically effective amount required for use in therapy varies with the nature of the condition being treated, the length of time desired to activate TLR activity, and the age/condition of the patient. The desired dose may be conveniently administered (e.g., intratumorally or peritumorally) in a single dose, or as multiple doses administered at appropriate intervals, for example as two, three, four or more sub-doses per day.

### II. IMMUNE CHECKPOINT THERAPIES (ICT)

Immune checkpoint therapies (ICT), also referred to as immune checkpoint inhibitors (ICIs) or immune checkpoint blockade (ICB), have provided significant improvements in the treatment of cancers. Some cancers can evade immunosurveillance by activation of immune checkpoint pathways that suppress antitumor immune responses. ICT can in many instances promote antitumor immune responses by interrupting co-inhibitory signaling pathways and promote immune-mediated elimination of tumor cells. In some preferred embodiments, flagellin or a flagellin derivative, such as CBLB502, is administered to a mammalian subject to treat a cancer in combination with both (i) an anti-PD1 antibody or an anti-PD-L1 antibody and (ii) an anti-CTLA4 antibody.

A variety of ICT can be used in various embodiments of the present invention. For example, the ICT may be an anti-CTLA4 antibody, such as for example ipilimumab (Schachter et al., 2017). Ipilimumab has been observed to reduce or prevent T-cell inhibition and promote the activation and proliferation of effector T cells.

In some embodiments, the ICT is an antibody that targets or selectively binds programmed death-1 (PD-1) or programmed death-ligand 1 (PD-L1). Examples of anti-PD-1 antibodies that can be used in various embodiments include pembrolizumab, nivolumab, and cemiplimab (Larkin, et al. 2015). Immune checkpoints, including PD-1 and CTLA-4, expressed on activated T cells can lead to inhibition of T-cell activation upon binding to their ligands on tumor cells/antigen-presenting cells. These interactions can be blocked using monoclonal antibodies, leading to the activation of T cells targeting tumor cells through the release of effector cytokines and cytotoxic granules (Liakou, et al., 2008) Anti-PD-L1 antibodies that may be used include, e.g., MPDL3280A, MEDI4736, and BMS-936559.

In some embodiments, the ICT comprises or consists of an anti-PD1 antibody or an anti-PDL1 antibody, such as, e.g., pembrolizumab, nivolumab, cemiplimab, avelumab, atezolizumab, durvalumab, nivolumab, REGN2810, MPDL3280A, MEDI4736, BMS-936558, SHR1210, KN035, IBI308, PDR001, BGB-A317, BCD-100, or JS001. The anti-CTLA-4 antibody may be ipilimumab or tremelimumab. In some preferred embodiments, the ICT comprises administering both: (i) an anti-PD1 antibody or an anti-PDL1 antibody, and (ii) an anti-CTLA-4 antibody to a mammalian subject to treat a cancer. In some embodiments, the ICT may comprise or consist of an anti-LAG3, anti-TIM-3, anti-VISTA, anti-TIGIT, anti-KIR, anti-CD47, anti-B7-H3, or anti-B7-H4 antibodies, ICOS and OX40 agonists; and IDO inhibitors.

Specific treatment regimens of an ICT that may be administered in combination with CBLB502 include, e.g., treatment doses ranging from 5 μg/day to 150 μg/day daily or every other day to a mammalian subject to treat a cancer, such as for example NSCLC, small cell lung cancer, head and neck squamous cell carcinoma, glioblastoma and other brain tumors, renal cell carcinoma, gastric adenocarcinoma, nasopharyngeal neoplasms, urothelial carcinoma, colorectal cancer, pleural mesothelioma, breast cancer, TNBC, esophageal neoplasms, multiple myeloma, gastric and gastroesophageal junction cancer, gastric adenocarcinoma, melanoma, Hodgkin lymphoma, hepatocellular carcinoma, lung cancer, mesothelioma, non-Hodgkin lymphoma, ovarian cancer, fallopian tube cancer, peritoneal neoplasms, or a solid malignancy. The ICT may be an OX40 inhibitor or an IDO inhibitors, a B7H3 inhibitor, or a B7H4 inhibitor.

### III. PHARMACEUTICAL PREPARATIONS

Pharmaceutical compositions of the present invention comprise an effective amount of a flagellin (e.g., CBLB5020) and/or an ICT, or additional agent dissolved or dispersed in a pharmaceutically acceptable carrier. In some embodiments, the CBLB502 and ICT are comprised in separate pharmaceutical preparations. Nonetheless, in some embodiments, it is anticipated that a flagellin (e.g., CBLB5020) and an ICT can be formulated in the same pharmaceutical preparation. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one of CBLB502 and/or an ICT will be known to those of skill in the art in light of the present disclosure, as exemplified by Remington: The Science and Practice of Pharmacy, 2^{nd} Ed., Lippincott Williams and Wilkins, 2005, incorporated herein by reference. Moreover, for animal (e.g., human) administration, it will be understood that preparations should typically meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., Mack Printing Company, 1990, pp. 1289-1329, incorporated herein by reference). Except insofar as any conventional carrier is incompatible with the active ingredient, its use in the pharmaceutical compositions is contemplated.

The flagellin (e.g., CBLB5020) and/or ICT may comprise different types of carriers depending on whether it is to be administered in solid, liquid or aerosol form, and whether it need to be sterile for such routes of administration as injection. The composition of the present disclosure can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The flagellin (e.g., CBLB5020) and/or ICT may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present disclosure is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The composition can be combined with the carrier in any convenient and practical manner, i.e., by solution, suspension, emulsification, admixture, encapsulation, absorption and the like. Such procedures are routine for those skilled in the art.

In a specific embodiment, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, a pharmaceutical lipid vehicle can comprise the flagellin (e.g., CBLB5020) and/or ICT, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In some embodiments, a flagellin (e.g., CBLB5020) and/or an ICT are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may, e.g., be formulated with an inert diluent or with an assimilable edible carrier, or they may be enclosed in hard- or soft-shell gelatin capsule, or they may be compressed into tablet.

A flagellin (e.g., CBLB5020) and/or ICT can be included a liquid formulations such as aqueous or oily suspensions, solutions, emulsions, syrups, and elixirs. The agents may also be formulated as a dry product for constitution with water or other suitable vehicle before use. Liquid preparations may contain additives such as suspending agents, emulsifying agents, nonaqueous vehicles and preservatives. Suspending agent may be sorbitol syrup, methyl cellulose, glucose/sugar syrup, gelatin, hydroxyethylcellulose, carboxymethyl cellulose, aluminum stearate gel, and hydrogenated edible fats. Emulsifying agents that can be used include lecithin, sorbitan monooleate, and acacia. Nonaqueous vehicles that can be used include edible oils, almond oil, fractionated coconut oil, oily esters, propylene glycol, and ethyl alcohol. Preservatives such as methyl or propyl p-hydroxybenzoate and sorbic acid can be included in the formulations.

Agents provided herein may also be formulated for parenteral administration such as by injection, intratumor injection, peritumoral injection, or continuous infusion. Formulations for injection may be in the form of suspensions, solutions, or emulsions in oily or aqueous vehicles, and may contain formulation agents including, but not limited to, suspending, stabilizing, and dispersing agents. The agent may also be provided in a powder form for reconstitution with a suitable vehicle including, but not limited to, sterile, pyrogen-free water.

As used herein, the term “parenteral” includes routes that bypass the alimentary tract. Specifically, the pharmaceutical compositions disclosed herein may be administered for example, but not limited to intravenously, intradermally, intramuscularly, intratumorally, peritumorally, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety).

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, intratumoral, peritumoral, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Ed., pages 1035-1038 and 1570-1580). Some variation in dosage will necessarily occur depending on the condition of the subject being treated. The person responsible for administration will, in any event, determine the appropriate dose for the individual subject.

The flagellin (e.g., CBLB5020) and/or ICT can be formulated as a depot preparation, which may be administered by implantation or by intratumoral or peritumoral injection. The agents may be formulated with suitable polymeric or hydrophobic materials (as an emulsion in an acceptable oil, for example), ion exchange resins, or as sparingly soluble derivatives (as a sparingly soluble salt, for example).

In some embodiments, the flagellin (e.g., CBLB5020) and/or ICT may be formulated for administration via various miscellaneous routes, for example, topical or transdermal administration, mucosal administration (intranasal, vaginal, etc.) and/or inhalation.

### IV. CANCERS

The therapies as described herein (e.g., CBLB502 in combination with an ICT, such as an anti-PD1 antibody and an anti-CTLA4 antibody) can be used to treat a variety of cancers. For example, in some embodiments, the cancer is NSCLC, small cell lung cancer, head and neck squamous cell carcinoma, glioblastoma, brain tumors, renal cell carcinoma, gastric adenocarcinoma, nasopharyngeal neoplasms, urothelial carcinoma, colorectal cancer, pleural mesothelioma, breast cancer, TNBC, esophageal neoplasms, multiple myeloma, gastric and gastroesophageal junction cancer, gastric adenocarcinoma, melanoma, Hodgkin lymphoma, hepatocellular carcinoma, lung cancer, mesothelioma, non-Hodgkin lymphoma, ovarian cancer, fallopian tube cancer, peritoneal neoplasms, or a solid malignancy.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### TLR5 Agonists Enhance Anti-Tumor Immunity and Overcome Therapy Resistance to Checkpoint Blockade

### In Vitro Characterization of 4T1 Tumor Cell Line

In Vitro NF-κB Activation of 4T1 Cells

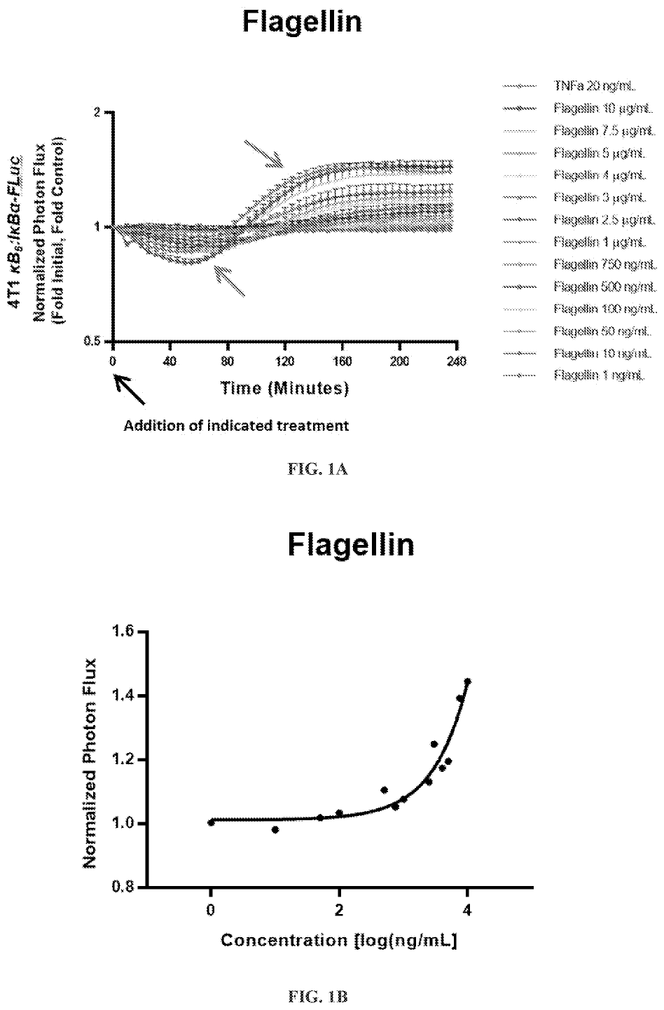

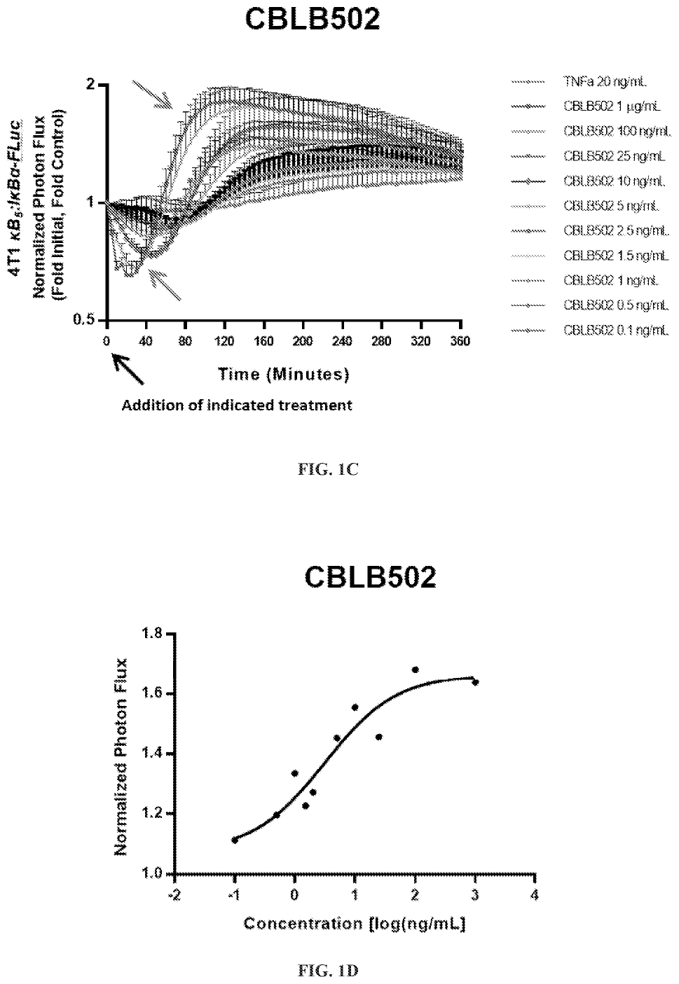

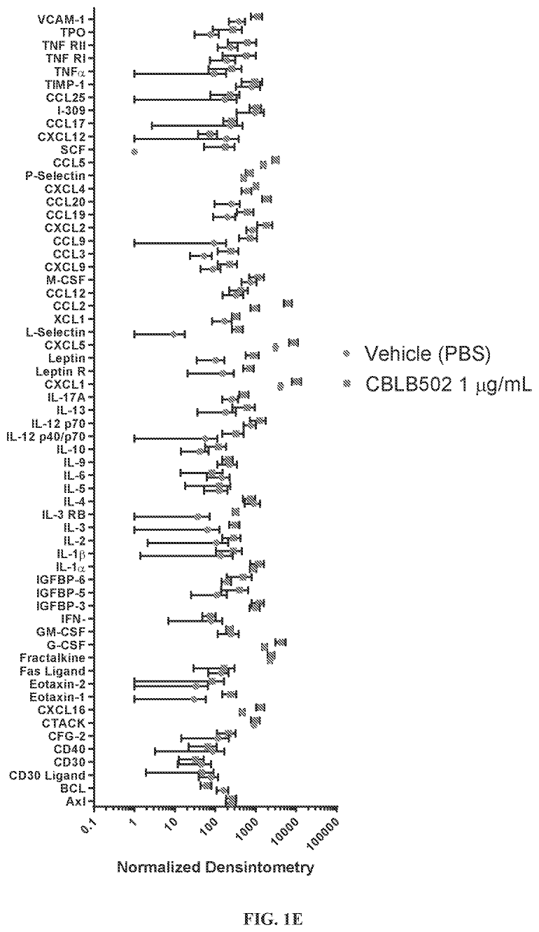

To evaluate flagella- and CBLB502-mediated NF-κB activation of 4T1 mammary carcinoma cells, 4T1 cells we stably transfected with a κB5:IκBa-FLuc transcriptional reporter comprised of a concatenated κB5 promoter region, followed by the bioluminescent IκBa-FLuc fusion reporter gene (Gross and Piwnica-Worms, 2005; Moss et al., 2008). This reporter provides a readout of endogenous ligand-induced IκB [symbol] degradation and production of new IκB [symbol] -FLuc fusion protein (Moss et al., 2008). In the cytoplasm, IκB [symbol] sequesters and inactivates NF-κB dimers. The binding of flagellin (or CBLB502) to TLR5 on the cell surface initiates TKK-mediated kinase activity, and the subsequent phosphorylation, ubiquination and targeting for proteasomal degradation of endogenous IκB [symbol] as well as the reporter fusion protein (Flentie et al., 2018; Moss et al., 2008). This resulted in a reduction of bioluminescent activity during the first 100 minutes in flagellin-treated cultures (FIG. 1A, red arrow) and the first 80 minutes in CBLB502-treated cultures (FIG. 1C, red arrow). The newly freed NF-κB dimers translocate to the nucleus and bind to the κB5 promoter region, initiating transcription and translation of new bioluminescent fusion proteins. This resulted in an increase in bioluminescent signal (FIGS. 1A and 1C, green arrows), which after a sufficient period of time, returned to a homeostatic state as previously observed with other NF-κB activating ligands (Flentie et al., 2018; Moss et al., 2012). Incubation of 4T1 cells with either flagellin or CBLB502 resulted in a concentration-dependent degradation and subsequent resynthesis of the IκBα-FLuc reporter fusion (FIGS. 1A and 1C). The half-maximal effective concentration (EC₅₀) for flagellin and CBLB502 were >10⁴ ng/mL and 3.1 ng/mL, respectively, in this cell line (FIGS. 1B and 1D), directly demonstrating the enhanced potency of CBLB502 for activating the NF-κB signaling pathway.

In Vitro Cytokine Profile of 4T1 Cells

CBLB502 activation of NF-κB pro-inflammatory signaling is mediated through TLR5 (Burdelya et al., 2008), a known activator of the innate immune system (Hayashi et al., 2001). Given that CLB502 is a potent activator of the NF-κB signaling in 4T1 carcinoma cells (FIG. 1C), we tested whether CBLB502 was sufficient to elicit immune stimulatory changes in the 4T1 cell cytokine profile. We measured protein arrays of 62 mouse cytokines secreted into conditioned media from 4T1 reporter cells in response to overnight treatment with CBLB502 (1 μg/mL). FIGS. 1E and 1F identified many up-regulated cytokines that activate innate immunity (in order of fold over control): SCF (176-fold), L-selecting (40-fold), IL3 and its receptor IL-3RB (11- and 9-fold respectively), Eotaxin-1 (8-fold), Leptin (9-fold), CCL20 (8-fold), CCL2 (7-fold), IL-12 p40/p70 (6 fold), CCL3 (5-fold), IGFBP-5 (4-fold), CCL19 (3-fold) and TNFα (3-fold), while some exert broader immune regulatory functions, such as VEGF (6-fold), G-CSF (3-fold), and IL-2 (3-fold) (Table 1). BCL (0.4-fold), CXCL-12 (0.5-fold), IL-6 (0.6-fold), CD40 (0.8-fold) and IL-4 (0.8-fold) showed decreased levels (FIGS. 1E and 1F, Table 1). Overall, incubation of CBLB502 (1 μg/mL) re-programed the cell cytokine signaling profile by upregulating many pro-inflammatory and immune activating cytokines (FIGS. 1E and 1F, Table 1).

TABLE 1
In vitro Cytokine Profile

| Cytokine | Vehicle (PBS)* | CBLB502 (1 μg/mL)* | Fold Difference |
|---|---|---|---|
| Axl | 254 | 258 | 1.0 |
| BCL | 158 | 62 | 0.4 |
| CD30 Ligand | 78 | 67 | 0.9 |
| CD30 | 40 | 35 | 0.9 |
| CD40 | 80 | 65 | 0.8 |
| CFG-2 | 116 | 214 | 1.8 |
| CTACK | 899 | 1,000 | 1.1 |
| CXCL16 | 466 | 1,330 | 2.8 |
| Eotaxin-1 | 30 | 239 | 8.1 |
| Eotaxin-2 | 33 | 82 | 2.5 |
| Fas Ligand | 134 | 164 | 1.2 |
| Fractalkine | 2,270 | 2,440 | 1.1 |
| G-CSF | 1,670 | 4,260 | 2.6 |
| GM-CSF | 247 | 228 | 0.9 |
| IFN-γ | 78 | 74 | 1.0 |
| IGFBP-3 | 983 | 1,190 | 1.2 |
| IGFBP-5 | 110 | 396 | 3.6 |
| IGFBP-6 | 194 | 489 | 2.5 |
| IL-1α | 880 | 1,150 | 1.3 |
| IL-1β | 136 | 279 | 2.1 |
| IL-2 | 106 | 283 | 2.7 |
| IL-3 | 28 | 308 | 11 |
| IL-3 RB | 37 | 319 | 8.7 |
| IL-4 | 905 | 728 | 0.8 |
| IL-5 | 126 | 124 | 1.0 |
| IL-6 | 144 | 83 | 0.6 |
| IL-9 | 227 | 209 | 0.9 |
| IL-10 | 41 | 120 | 2.9 |
| IL-12 p40/p70 | 57 | 324 | 5.7 |
| IL-12 p70 | 746 | 1230 | 1.7 |
| IL-13 | 178 | 610 | 3.4 |
| IL-17A | 255 | 530 | 2.1 |
| CXCL1 | 4,100 | 10,600 | 2.6 |
| Leptin R | 154 | 692 | 4.5 |
| Leptin | 102 | 870 | 8.6 |
| CXCL5 | 3,080 | 8,880 | 2.9 |
| L-Selectin | 9 | 370 | 40 |
| XCL1 | 168 | 328 | 2.0 |
| CCL2 | 975 | 6,390 | 6.6 |
| CCL12 | 325 | 429 | 1.3 |
| M-CSF | 752 | 1,139 | 1.5 |
| CXCL9 | 88 | 229 | 2.6 |
| CCL3 | 52 | 244 | 4.6 |
| CCL9 | 93 | 726 | 7.8 |
| CXCL2 | 831 | 1,820 | 2.2 |
| CCL19 | 200 | 608 | 3.0 |
| CCL20 | 252 | 1,910 | 7.6 |
| CXCL4 | 611 | 1020 | 1.7 |
| P-Selectin | 508 | 703 | 1.4 |
| CCL5 | 1,550 | 3,130 | 2.0 |
| SCF | 1 | 176 | 180 |
| CXCL12 | 141 | 74 | 0.5 |
| CCL17 | 237 | 251 | 1.1 |
| I-309 | 963 | 1,020 | 1.1 |
| CCL25 | 170 | 233 | 1.4 |
| TIMP-1 | 804 | 949 | 1.2 |
| TNFα | 93 | 255 | 2.7 |
| TNF RI | 191 | 582 | 3.0 |
| TNF RII | 237 | 620 | 2.6 |
| TPO | 76 | 270 | 3.5 |
| VCAM-1 | 382 | 1,100 | 2.9 |
| VEGF-A | 12 | 73 | 6.3 |

*Normalized densitometry

Treatment of ICT Refractory 4T1 Carcinomas

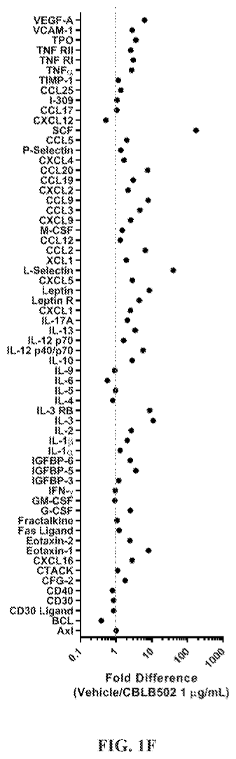

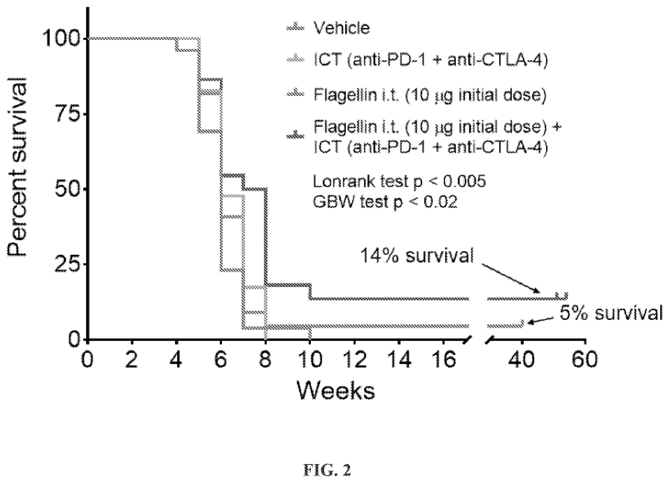

Next, the inventors investigated whether administration of flagellin or CBLB502 could elicit antitumor responses in a syngeneic triple negative breast cancer 4T1 tumor model in vivo. Mammary cell carcinomas were generated in BALB/c mice (5-6 weeks old) by orthotopic injection of 4T1 FUGW-FL tumor cells into the right fourth mammary fat pad. Tumor progression of each mouse was assessed weekly using bioluminescence imaging and caliper measurements of tumor volume (FIG. 2). Bioluminescent signal was detected one week post orthotopic injection, confirming successful tumor implantation. Tumors were palpable and displayed strong bioluminescence signal two weeks post orthotopic injection, indicating robust tumor growth. Mice were then randomized into four different treatment controls: vehicle control, ICT (anti-PD-1 and anti-CTLA-4), flagellin or CBLB502 treatment, and flagellin or CBLB502 in combination with ICT treatment at the indicated dose (Table 2) and delivery method (Table 3).

TABLE 2
Treatment Doses and Delivery Routes

| | Initial dose and | Subsequent dose and |
|---|---|---|
| Treatment | delivery site | delivery site |
| Flagellin | 10 μg i.t. or i.p. | 2 μg (8x) i.t. or i.p. |
| CBLB502 High Dose | 10 μg i.t. or i.p. | 2 μg (8x) i.t. or i.p. |
| CBLB502 Low Dose | 1 μg i.t. or i.p. | 0.5 μg (8x) i.t. or i.p. |
| anti-CTLA-4 (9D9) | 200 μg i.p. | 100 μg i.p. (3x) |
| anti-PD-1 (RPM1-1) | 200 μg i.p. | 100 μg i.p. (3x) |

TABLE 3
Murine 4T1 Carcinoma Experiments.

| 4T1 Murine | | | | | |
|---|---|---|---|---|---|
| Carcinoma | | | | | |
| Experiments | Innate immune activating treatment | | | | ICT (i.p.) |
| Experiment 1 | Flagellin (i.t.) | | | | With or |
| | | | | | without |
| Experiment 2 | Flagellin (i.t.) | CBLB502 | | | With or |
| | | High Dose (i.t.) | | | without |
| Experiment 3 | Flagellin (i.t.) | CBLB502 | CBLB502 | | With or |
| | | High Dose (i.t.) | Low Dose (i.t.) | | without |
| Experiment 4 | Flagellin (i.t.) | CBLB502 | CBLB502 | | With or |
| | | High Dose (i.t.) | Low Dose (i.t.) | | without |
| Experiment 5 | | | CBLB502 | CBLB502 | With or |
| | | | Low Dose (i.t.) | Low Dose (i.p.) | without |
| Experiment 6 | | | CBLB502 | CBLB502 | With or |
| | | | Low Dose (i.t.) | Low Dose (i.p.) | without |
| Experiment 7 | No Treatment | No Treatment | No Treatment | No Treatment | No |
| (Re-challenge | | | | | Treatment |
| experiment) | | | | | |

### Intratumoral Injection (i.t.); Intraperitoneal Injection (i.p.).

Flagellin Treatment

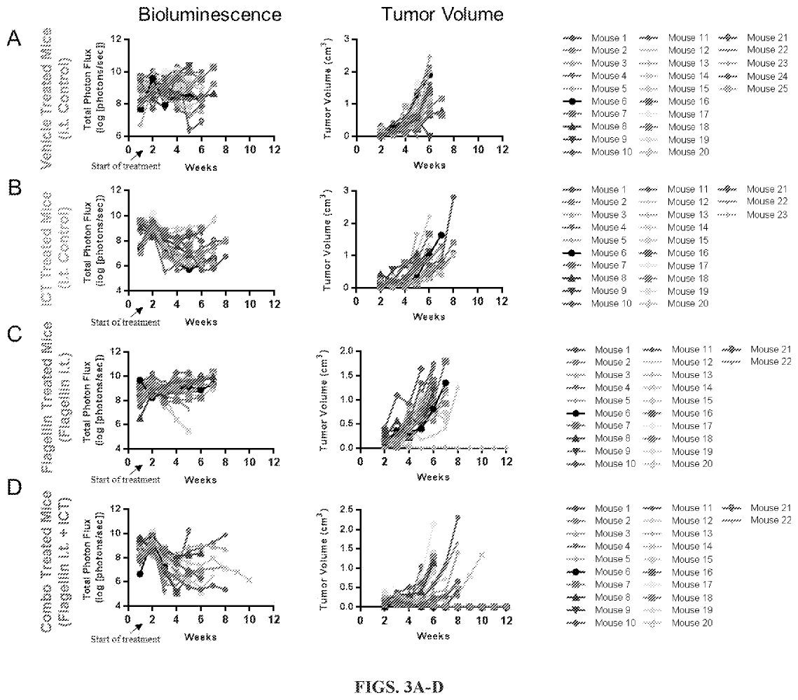

The overall survival combined from four independent experiments is shown in FIG. 2. All mice under vehicle control¹ (n=26) or ICT only treatment (n=23) died by the end of the study, confirming the ICT refractory status of the 4T1 breast cancer model. Tumor-free mice were observed in intratumoral flagellin only treatment (10 μg/mouse initial dose) (n=22) (one survivor, p=0.2, Log-rank test) and in intratumoral flagellin+ICT treatment (n=22) (three survivors, p=0.01, Log-rank test) (FIG. 2). Mice under vehicle control and flagellin only treatment controls showed steady bioluminescent signal with increase in tumor volume (FIGS. 3A and 3C). Interestingly, ICT-treated mice (ICT only or flagellin+ICT-treated mice) showed a sharp decreased in bioluminescent signaling one week post initiation of treatment (FIGS. 3B and 3D, left panel); however, the decrease in bioluminescent signaling was not accompanied by an overall decrease in tumor volume (FIGS. 3B and 3D, right panel). At least two possibilities could account for this observation: tumor cell death accompanied by an increased in cellular immune infiltrate or selective loss or silencing of the bioluminescent cassette. Though not mutually exclusive, these results overall pointed to an ICT-induced remodeling in the tumor microenvironment with abundant immune cell infiltrates, which was not predictive of survival per se.

CBLB502 Treatment

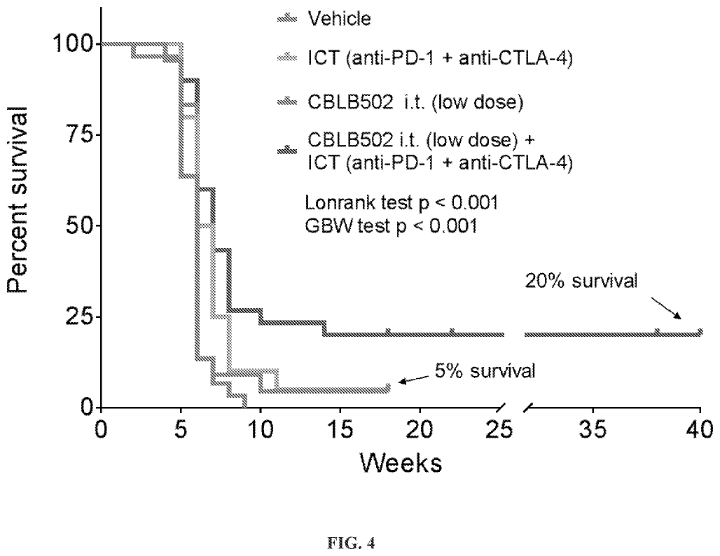

Given that CBLB502 showed greater potency and efficacy for activation of NF-κB signaling than flagellin (FIGS. 1C-D compared with FIGS. 1A-B) and that CBLB502 stimulated innate immune activating cytokines (FIGS. 1E-1F; Table 1), the inventors tested whether intratumoral treatment with CBLB502 (low dose) could prompt a stronger antitumor response than intratumoral treatment with flagellin. The overall survival combined from four independent experiments (Table 3) is shown in FIG. 4. Interestingly, one mouse from the vehicle control group (n=22) was tumor-free by the end of the experiment. This mouse showed comparable bioluminescent signal during the first two weeks post tumor implantation, ruling out the possibility that not enough cells were implanted for tumor growth (FIG. 5A—Mouse 25). However, it is noteworthy that no palpable tumor was detected during the course of the experiment making it likely that the mouse sequestered the tumor before tumor cells could establish significant growth (FIG. 5A—Mouse 25). Treatment with CBLB502 alone (low dose) (n=30) resulted in steady bioluminescent signal and tumor growth with no survivors. ICT treatment alone (n=20) resulted in one tumor-free mouse (FIG. 5B—Mouse 23) and one mouse that at first responded to treatment, but later slowly developed a tumor (FIG. 5B—Mouse 7). Consistent with previous results, ICT-treated mice (ICT only or CBLB502 (low dose)+ICT treatments) showed a sharp decrease in bioluminescent signal one week post treatment initiation (FIG. 5B, FIG. 5D, FIG. 3B, and FIG. 3D), consistent with a significant initial loss of 4T1 tumor cells. Most importantly, CBLB502 (low dose)+ICT treatment resulted in 20% tumor-free mice (n=30) (p=0.001, Log-rank test; p=0.001, Gehan-Breslow-Wilcoxon test) (FIG. 4).

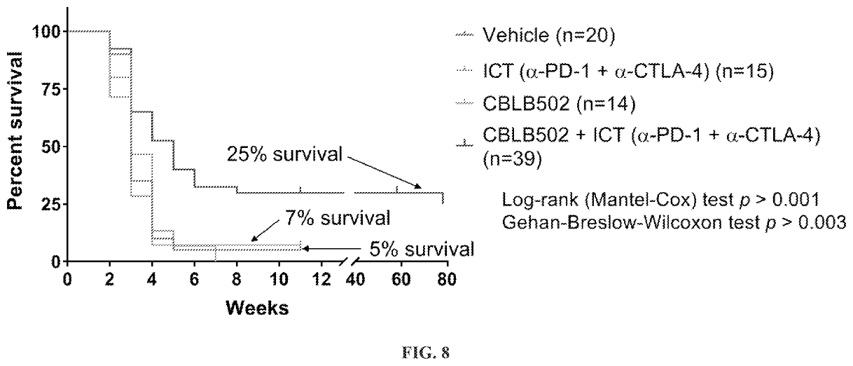

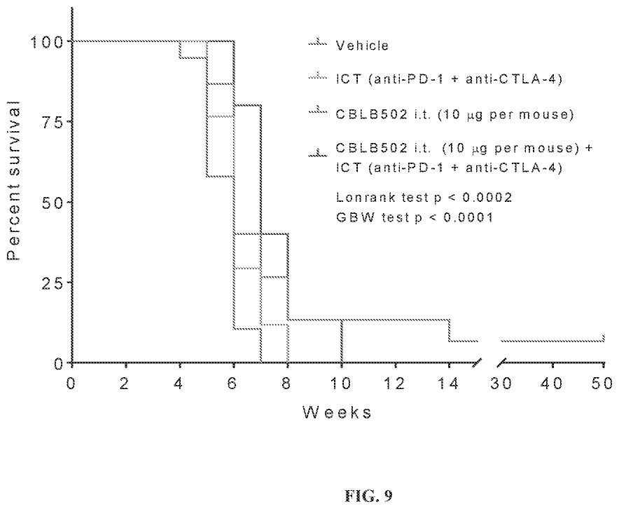

A higher intratumoral CBLB502 dose (CBLB502 high dose) was tested, which is comparable to the dose administered to mice in the flagellin treatment cohort (Table 2). In three independent experiments (Table 3), treatment with CBLB502 (high dose) resulted in one tumor free mouse in the CBLB502 (high dose) only treatment (n=15) with no survivors in any of the other treatments (vehicle (n=19), ICT (n=17), CBLB502 (high dose)+ICT (n=15)) (FIG. 9). Interestingly, one additional mouse in the CBLB502 (high dose) only treatment showed delayed tumor growth (FIG. 1C—Mouse 12). The lone-survivor (FIG. 1C—Mouse 13) showed a statistically detectable difference in survival from its vehicle control (p=0.01, Log-rank test; p=0.02, Gehan-Breslow-Wilcoxon test).

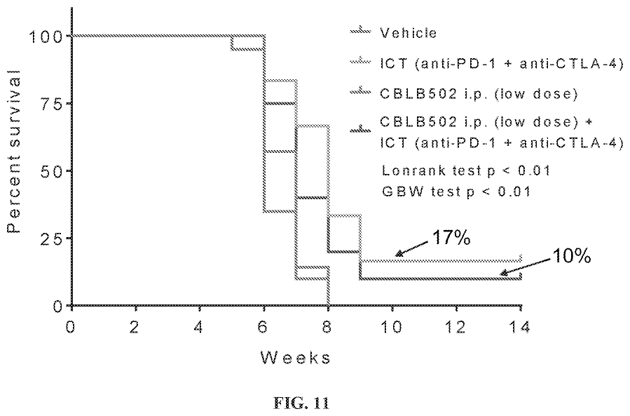

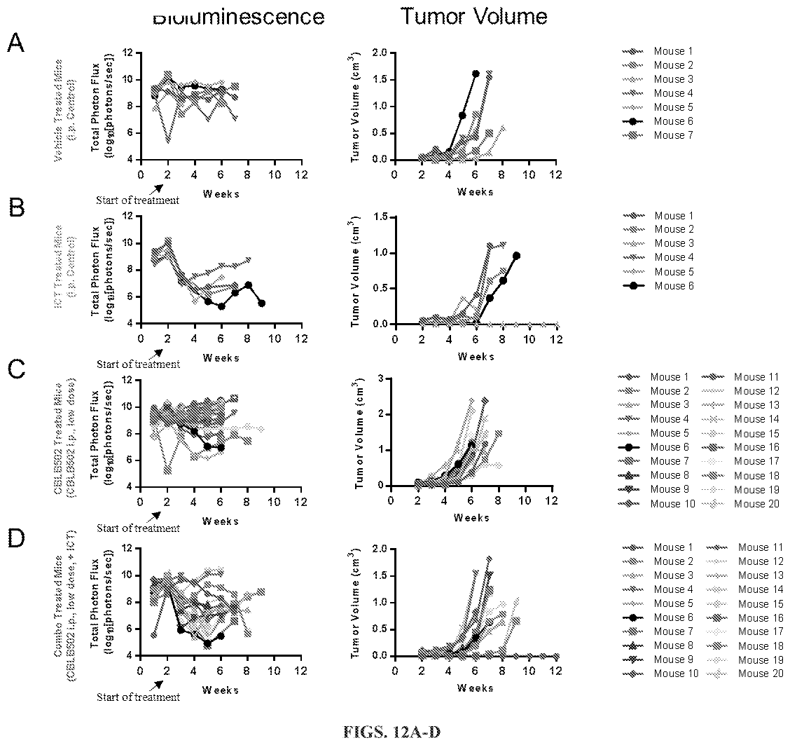

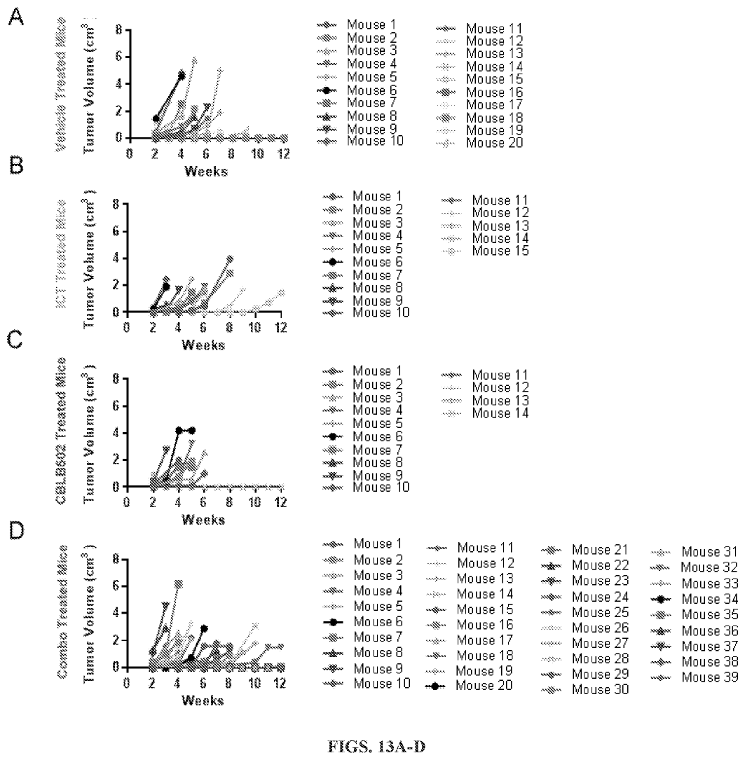

Whether systemic delivery of CBLB502 (low dose) via intraperitoneal (i.p.) injections could elicit a similar response to intra-tumoral delivery of CBLB502 (low dose) was tested. It was observed in two independent experiments (Table 3) that i.p. delivered CBLB502 (low dose)+ICT treatment resulted in 10% long-term survivors (n=20) (FIG. 12 and FIGS. 13A-D). Whereas CBLB502 alone (low dose, i.p. delivery, n=20) and vehicle control (n=7) resulted in no long-term survivors (FIG. 11 and FIGS. 12A-D), ICT-treated mice (n=6) resulted in one long-term survivor in this experiment (FIG. 11 and FIGS. 12A-D).

### Tumor Re-Challenge Experiments and Immune Memory

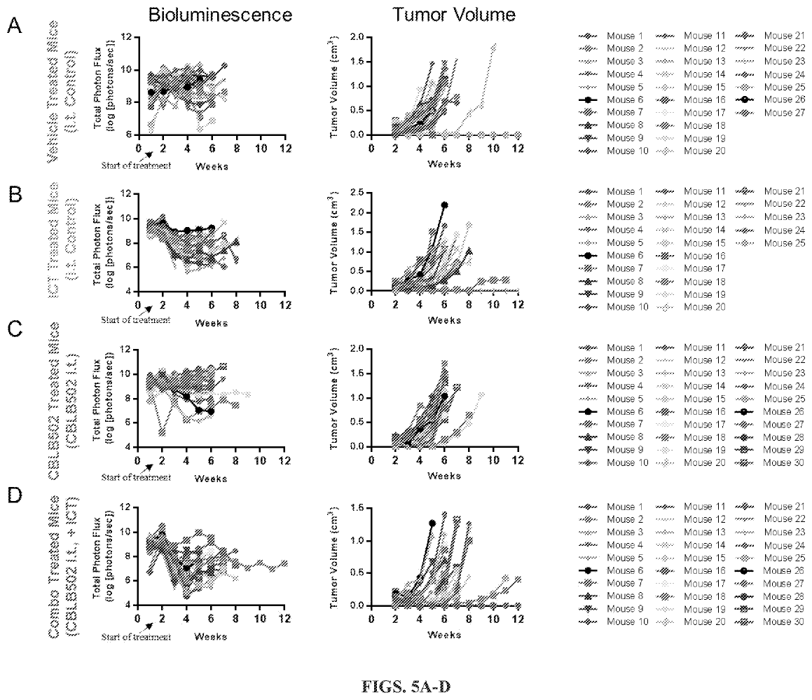

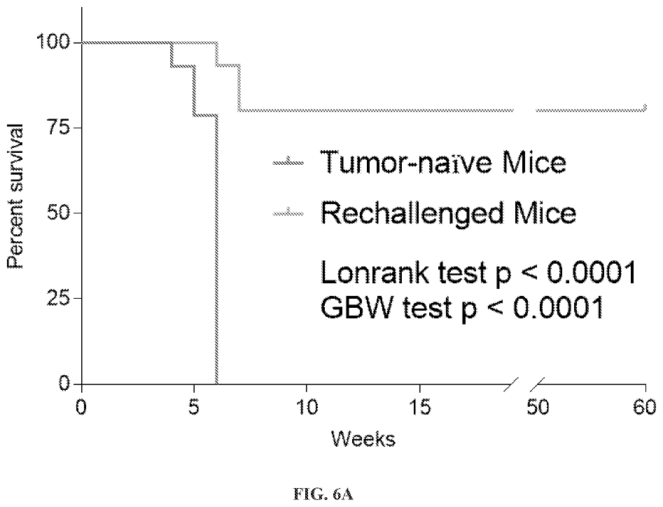

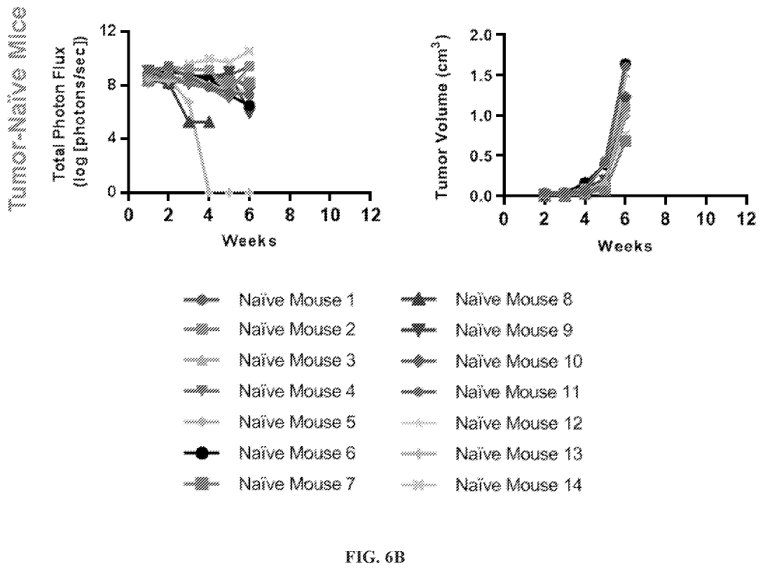

Mice that showed complete tumor regression were re-challenged by orthotopic injections of 4T1 FUGW-FL cells into the opposite (left) fourth mammary fat pad without any additional therapy. FIG. 6A shows overall survival from the re-challenge experiment. All untreated tumor-naïve, tumor bearing and aged-matched control mice died by week six confirming the aggressive potential of the tumor cell cohort, whereas 80% of re-challenged mice were tumor-free for at least 60 weeks post tumor implantation (FIG. 6A). One re-challenged mouse was excluded from the survival curve shown in FIG. 6A because the cause of death was unrelated to tumor implantation². Furthermore, this mouse showed no bioluminescent signal or palpable tumor during the first three weeks of the experiment (FIG. 6C, vehicle mouse: FIG. 5A—Mouse 22, Table 4), indicating that the mouse likely rejected 4T1 FUGW-FL tumor implantation. Overall, these results indicated that the observed curative effects were likely due to acquired memory for anti-tumor immunity.

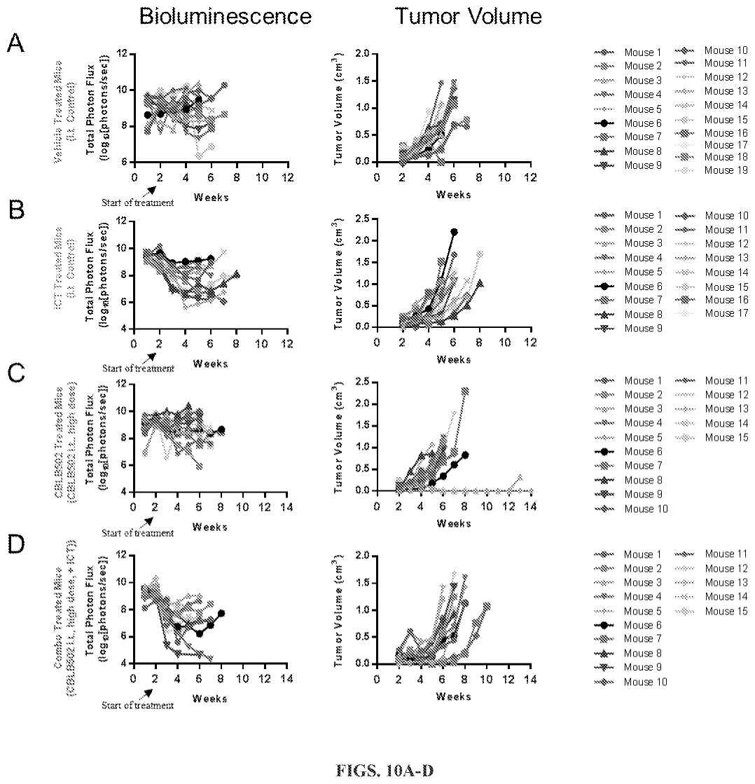

TABLE 4
Re-challenge Experiment

| | | Time Alive | |
|---|---|---|---|
| | | Post-tumor | Outcome |
| | | Implantation | (Post-re- |
| Original cohort | Mouse I.D. | (weeks) | challenge) |
| Tumor-Naïve | FIG. 6B-Naïve Mouse 1 | 51* | Dead W6 |
| Tumor-Naïve | FIG. 6B-Naïve Mouse 2 | 51* | Dead W6 |
| Tumor-Naïve | FIG. 6B-Naïve Mouse 3 | 51* | Dead W6 |
| Tumor-Naïve | FIG. 6B-Naïve Mouse 4 | 51* | Dead W5 |
| Tumor-Naïve | FIG. 6B-Naïve Mouse 5 | 51* | Dead W5 |
| Tumor-Naïve | FIG. 6B-Naïve Mouse 6 | 22* | Dead W6 |
| Tumor-Naïve | FIG. 6B-Naïve Mouse 7 | 22* | Dead W6 |
| Tumor-Naïve | FIG. 6B-Naïve Mouse 8 | 22* | Dead W4 |
| Tumor-Naïve | FIG. 6B-Naïve Mouse 9 | 22* | Dead W6 |
| Tumor-Naïve | FIG. 6B-Naïve | 22* | Dead W6 |
| | Mouse 10 | | |
| Tumor-Naïve | FIG. 6B-Naïve | 18* | Dead W6 |
| | Mouse 11 | | |
| Tumor-Naïve | FIG. 6B-Naïve | 18* | Dead W6 |
| | Mouse 12 | | |
| Tumor-Naïve | FIG. 6B-Naïve | 18* | Dead W6 |
| | Mouse 13 | | |
| Tumor-Naïve | FIG. 6B-Naïve | 18* | Dead W6 |
| | Mouse 14 | | |
| Vehicle | FIG. 5A-Mouse 25 | 18 | Alive |
| Flagellin | FIG. 3C-Mouse 13 | 40 | Alive |
| CBLB502 high dose | FIG. 10C-Mouse 5 | 51 | Dead W7 |
| ICT | FIG. 5B-Mouse 23 | 22 | Dead W6 |
| ICT | FIG. 12B-Mouse 3 | 18 | Alive |
| Flagellin (i.t.) + ICT | FIG. 3D-Mouse 2 | 54 | Alive |
| Flagellin (i.t.) + ICT | FIG. 3D-Mouse 6 | 51 | Alive |
| Flagellin (i.t.) + ICT | FIG. 3D-Mouse 7 | 51 | Alive |
| CBLB502 low dose | FIG. 5D-Mouse 1 | 40 | Dead W6 |
| (i.t.) + ICT | | | |
| CBLB502 low dose | FIG. 5D-Mouse 4 | 40 | Alive |
| (i.t.) + ICT | | | |
| CBLB502 low dose | FIG. 5D-Mouse 10 | 38 | Alive |
| (i.t.) + ICT | | | |
| CBLB502 low dose | FIG. 5D-Mouse 19 | 22 | Alive |
| (i.t.) + ICT | | | |
| CBLB502 low dose | FIG. 5D-Mouse 22 | 18 | Alive |
| (i.t.) + ICT | | | |
| CBLB502 low dose | FIG. 5D-Mouse 26 | 18 | Alive |
| (i.t.) + ICT | | | |
| CBLB502 low dose | FIG. 12D-Mouse 1 | 22 | Alive |
| (i.t.) + ICT | | | |
| CBLB502 low dose | FIG. 12D-Mouse 5 | 22 | Alive |
| (i.t.) + ICT | | | |

*Tumor-naïve mice are age-matched mice that were part of the original cohort of mice, but were not implanted with tumor cells during the original experiments, W = week.

### In Vivo Cytokine Profile

Characterization of Peripheral Blood Cytokine Profile

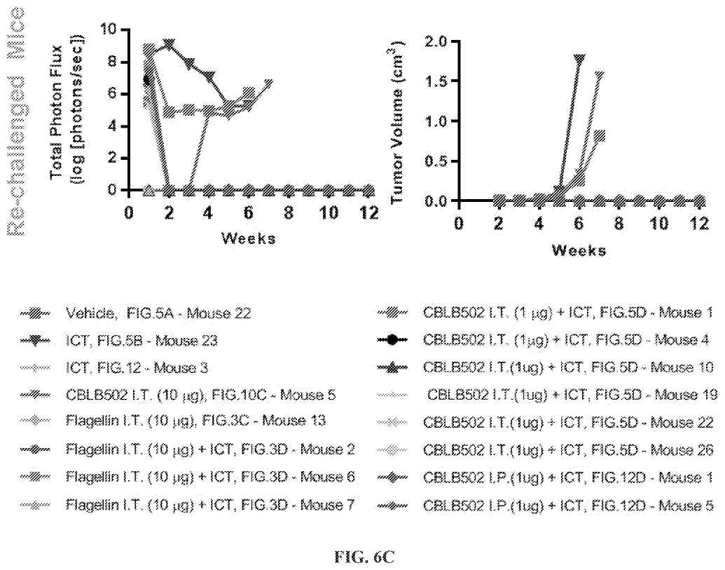

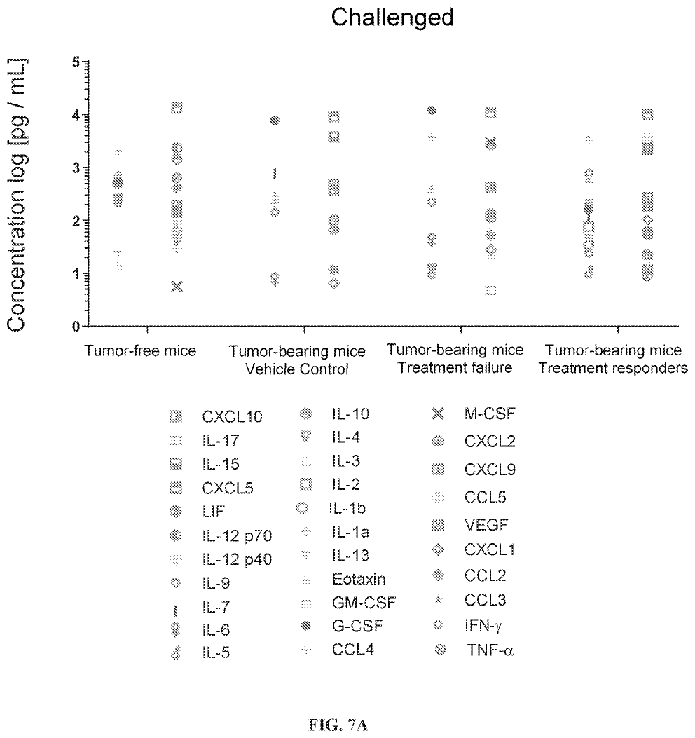

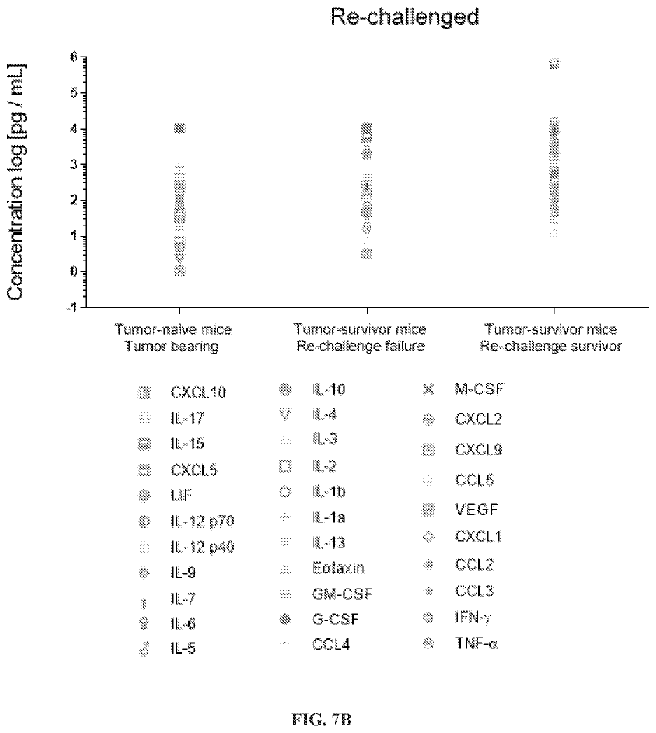

To begin to explore the mechanisms of response and characterize changes elicited by ICT and CBLB502 therapies alone or in combination, the inventors assayed 32 peripheral blood-borne cytokines from aged matched tumor-free mice (healthy mice) and 4T1 FUGW-FL tumor-bearing mice under the treatment cohorts: tumor-bearing, vehicle control; tumor-bearing treatment failure; and tumor-bearing, treatment responders, during weeks 5 to 7 (FIG. 7A). First, it is noteworthy that levels of granulocyte-colony stimulating factor (G-CSF), a cytokine involved in the proliferation and differentiation of granulocytes and neutrophils (Hayashi et al., 2002) and associated with poorer survival in cervical, non-small cell lung cancer, colon, melanoma and skin cancers (Matsuda et al., 2009; Fukutomi et al., 2012; Stathopoulos et al., 2011; Aliper et al., 2014) increased 15-fold in tumor-bearing-vehicle treated non-surviving mice and 23-fold in those tumor-bearing mice that failed to respond to treatment when compared with healthy tumor-free mice (FIG. 7A, Table 5). In contrast, tumor-bearing mice that responded to treatment showed a 3-fold decrease in G-CSF. Second, tumor-bearing mice that responded to treatment showed upregulation of innate immune activating cytokines compared to tumor-free controls: M-CSF (560-fold), CXCL2 (18-fold), IL-15 (15-fold), IL-13 (9-fold), CCL3 (5 fold), IL-9 (3-fold), and CXCL1 (2-fold) (FIG. 7A, Table 5). Among these cytokines, IL-13, IL-9, CCL3 and CXCL1 showed higher levels in tumor-bearing mice that responded to treatment compared to those mice that failed treatment.

TABLE 5
In vivo cytokine profile: treatment response.

| | Naïve | | | | | | |
|---|---|---|---|---|---|---|---|
| | Tumor-free | Vehicle | Fold | Failed | Fold | Long-term | Fold |
| | mice | Control | Change | Treatment | Change | survivors | change |
| Cytokine | (pg/mL) | (pg/mL) | Veh. | (pg/mL) | Failed | (pg/mL) | Survivors |
| G-CSF | 524 | 7,750 | 15 | 12,100 | 23 | 161 | 0.3 |
| GM-CSF | | | | | | 53 | |
| Eotaxin | 805 | 308 | 0.4 | 407 | 0.5 | 607 | 0.8 |
| IL-13 | 23 | | | | | 217 | 9 |
| IL-1α | 1,900 | 215 | 0.1 | 3,720 | 5.0 | 3,420 | 2 |
| IL-1β | 504 | | | | | 35 | 0.1 |
| IL-2 | | | | | | 75 | |
| IL-3 | 14 | | | | | | |
| IL-4 | 252 | | | 12 | 0.05 | | |
| IL-5 | 239 | | | 11 | 0.05 | 11 | 0.05 |
| IL-6 | 601 | 8 | 0.01 | 43 | 0.1 | 178 | 0.3 |
| IL-7 | | 928 | | | | 148 | |
| IL-9 | 247 | 142 | 0.6 | 223 | 0.9 | 786 | 3 |
| IFN-γ | | | | | | 24 | |
| IL-10 | 1,450 | | | 134 | 0.1 | 23 | 0.02 |
| IL-12 p40 | | | | | | 3,700 | |
| IL-12 p70 | 2,340 | | | 114 | 0.05 | 55 | 0.02 |
| LIF | | 67 | | | | 61 | |
| CXCL5 | 13,600 | 9,300 | 0.7 | 11,100 | 0.8 | 10,200 | 0.7 |
| IL-15 | 148 | 3,790 | 26 | | | 2,230 | 15 |
| IL-17 | 58 | | | 5 | 0.1 | | |
| CXCL10 | 194 | 370 | 2 | 417 | 2 | 186 | 1 |
| CXCL1 | 64 | 7 | 0.1 | 28 | 0.4 | 103 | 2 |
| CCL2 | 412 | 12 | 0.03 | 52 | 0.1 | 60 | 0.1 |
| CCL3 | 39 | | | | | 189 | 5 |
| CCL4 | 31 | 84 | 3 | 56 | 2 | 58 | 2 |
| M-CSF | 6 | | | 3,020 | 536 | 3,170 | 561 |
| CXCL2 | 157 | 103 | 0.7 | 2,700 | 17 | 2,890 | 18 |
| CXCL9 | 145 | 472 | 3 | 427 | 3 | 269 | 2 |
| CCL5 | 103 | 11 | 0.1 | 23 | 0.2 | 20 | 0.2 |
| VEGF | | | | | | 12 | |
| TNF-α | 642 | | | | | 9 | 0.01 |

Cytokines highlighted in Bold showed higher levels in responsive mice compared to mice that failed therapies.

Characterization of Peripheral Blood Cytokine Profile of Re-Challenged Mice

Tumor-re-challenged and tumor-naïve mice were assayed three weeks post 4T1 FUGW-FL implantation. Mice that were tumor-free for at least 60 weeks post re-challenge (tumor survivor, re-challenge survivor) revealed a distinctive cytokine profile from those mice that were re-challenged, but developed tumors (tumor survivor, re-challenge failure) (FIG. 7B). Interestingly, similar to previous results (FIG. 7A), levels of G-CSF were lower in long-term survivors (tumor survivor, re-challenge survivor) (18-fold decreased) when compared with tumor-naïve, tumor-bearing mice (non-survivors) (FIG. 7B, Table 6). Mice that were re-challenged, but did not survive (tumor survivor, re-challenge failure) showed similar levels of G-CSF to tumor-naïve, tumor-bearing mice at this time point. Four profiles emerged from this analysis. First, cytokines that were upregulated in both re-challenge failure mice and re-challenge survivor mice, but with a far greater increase in re-challenge survivors: IL-15 (173-fold compared with 20,000-fold), LIF (5-fold compared with 420-fold), CXCL1 (3-fold compared with 120-fold), IL-2 (7-fold compared with 260-fold), IL-7 (120-fold compared with 4,400-fold), IL-12 p70 (10-fold compared with 250-fold), CCL4 (2-fold compared with 57-fold), Eotaxin (3-fold compared with 27-fold), CCL3 (3-fold compared with 20-fold), IL-1α (4-fold compared with 22-fold), IL-10 (55-fold compared with 212-fold), IL-1β (3-fold compared with 6-fold), CXCL2 (8-fold compared with 17-fold), IL-4 (17-fold compared with 35-fold), IL-9 (4-fold compared with 7-fold), and M-CSF (40-fold compared with 60-fold) (Table 6). Second, cytokines that were downregulated in both groups, but to a greater extent in the re-challenge failure cohort: IL-3 (0.4 compared with 0.7 decrease), IL-5 (0.3 compared with 0.5 decrease), and CXCL10 (0.3 compared with 0.5 decrease) (Table 5). Third, cytokines that showed differential regulation between the two cohort: IL-12 p40 (0.4 decrease compared with 2-fold increase), TNFα (0.5 decrease compared with a 2-fold increase), IL-6 (0.7 decrease compared with 2-fold increase) (Table 6). Fourth, cytokines that did not change in one population, but did in another: re-challenge survivor mice upregulated IL-13 (53-fold), CXCL9 (12-fold), IFN-7 (8-fold), CCL5 (3-fold), whereas re-challenge failure mice did not upregulate these cytokines greater than 2-fold. CXCL5 showed a 0.8 decrease in long-term survivors, but no change in mice that failed to survive. IL-17 showed a 1.5-fold increase in mice that failed to survive, but no change in long-term survivors (Table 6). Taken together, these results showed a distinctive adaptive immune-activating cytokine profile in those mice that survived the re-challenge experiment.

TABLE 6
In vivo cytokine profile: re-challenge experiment.

| | Tumor- | Failed | Fold-change | Long-term | Fold-change |
|---|---|---|---|---|---|
| | naïve | survivors | Failed | survivors | Long-term |
| Cytokine | (pg/mL) | (pg/mL) | survivors | (pg/mL) | survivors |
| G-CSF | 10,100 | 10,000 | 1.0 | 540 | 0.1 |
| GM-CSF | 490 | 400 | 0.8 | 1,100 | 2 |
| Eotaxin | 44 | 130 | 3 | 1,200 | 27 |
| IL-13 | 16 | 22 | 1 | 848 | 53 |
| IL-1α | 870 | 3,400 | 4 | 19,000 | 22 |
| IL-1β | 58 | 170 | 3 | 360 | 6 |
| IL-2 | 7 | 46 | 7 | 1,800 | 260 |
| IL-3 | 20 | 7 | 0.4 | 14 | 0.7 |
| IL-4 | 2 | 41 | 18 | 81 | 35 |
| IL-5 | 98 | 30 | 0.3 | 50 | 0.5 |
| IL-6 | 65 | 44 | 0.7 | 120 | 2 |
| IL-7 | 3 | 310 | 120 | 11,000 | 4,400 |
| IL-9 | 460 | 1,900 | 4 | 3,400 | 7 |
| IFN-γ | 110 | 130 | 1 | 930 | 8 |
| IL-10 | 38 | 2,100 | 55 | 8,000 | 213 |
| IL-12 p40 | 451 | 195 | 0.4 | 1,000 | 2 |
| IL-12 p70 | 5 | 51 | 10 | 1,200 | 250 |
| LIF | 38 | 180 | 5 | 16,000 | 420 |
| CXCL5 | 11,000 | 11,000 | 1.0 | 8,900 | 0.8 |
| IL-15 | 34 | 5,800 | 170 | 650,000 | 19,000 |
| IL-17 | 34 | 52 | 2 | 32 | 0.9 |
| CXCL10 | 450 | 140 | 0.3 | 210 | 0.5 |
| CXCL1 | 31 | 77 | 3 | 3,800 | 120 |
| CCL2 | 120 | 240 | 2 | 260 | 2 |
| CCL3 | 92 | 240 | 3 | 1,800 | 20 |
| CCL4 | 38 | 94 | 3 | 2,200 | 57 |
| M-CSF | 47 | 1,900 | 40 | 2,900 | 61 |
| CXCL2 | 260 | 2,200 | 8 | 4,500 | 17 |
| CXCL9 | 220 | 250 | 1 | 2,600 | 12 |
| CCL5 | 38 | 45 | 1 | 120 | 3 |
| VEGF | 1 | 3 | 3 | 620 | 590 |
| TNF-α | 35 | 16 | 0.5 | 62 | 2 |

Cytokines highlighted in Bold showed higher levels in long-term survivor mice mice that failed therapies.

Treatment of ICT Refractory B16-F10 Melanoma Tumor

We further investigated whether combination treatment of CBLB502 and ICT could also elicit antitumor responses in a poorly immunogenic tumor such as B16-F10 melanoma tumor model. Melanoma tumors were generated in C57BL/6J (6-9 weeks old) by subcutaneous injection of B16-F10 tumor cells into the right dorsal flank. Three days after tumor implantation, mice were randomized into four different treatment controls: vehicle control, ICT (anti-PD-1 and anti-CTLA-4), CBLB502 treatment, and CBLB502 in combination with ICT treatment at the indicated dose and delivery method (Table 7). Tumor progression of each mouse was assessed bi-weekly using caliper measurements of tumor volume (FIG. 13A-D).

TABLE 7
B16-F10 Melanoma Experiments

| B16-F10 | Vehicle | Innate immune | |
|---|---|---|---|
| Melanoma | Control | activating | |
| Experiments | (i.t. and i.p) | treatment control | ICT (i.p.) |
| Experiment 10 | PBS | CBLB502 (i.t) | Only with ICT |
| Experiment 13 | PBS | CBLB502 (i.t) | With or without |
| Experiment 15 | PBS | CBLB502 (i.t.) | With or without |
| Experiment 19 | PBS | CBLB502 (i.t.) | With or without |

Intratumoral Injection (i.t.); Intraperitoneal Injection (i.p.).

The overall survival combined from four independent experiments is shown in FIG. 8. Out of four independent experiments, one vehicle control mouse out of twenty mice did not develop a palpable tumor during the duration of the experiment. It is possible that a failure to inject appropriate number of tumor cells precluded tumor implantation and subsequent tumor growth. All ICT only treatment mice (n=15) died by the end of the study, confirming the ICT refractory status of the B16-F10 melanoma tumor model without the addition of a vaccine. Only one mouse in the CBLB502 treatment (n=14), did not develop a tumor by the end of the study. However, survival curves showed no detectable significant difference with vehicle control group (p=0.5, Log-rank test; p=0.3, Gehan-Breslow-Wilcoxon test). Combination treatment with CBLB502 and ICT resulted in 25% tumor-free mice (n=39) by week 78 of the study (p=0.001, Log-rank test; p=0.003, Gehan-Breslow-Wilcoxon test). As shown in FIG. 4 and FIG. 8, combination treatment with CBLB502 and ICT enhanced survival in vivo from two independent ICT refractory tumor models.

4T1 mammary carcinoma is a robust murine model to study human triple negative breast cancer, which is highly invasive, metastatic and resistant to immune check point therapies (Dexter et al., 1978; Aslakson and Miller, 1992). Herein it was observed that: 1) the successful treatment of established ICT-refractory murine 4T1 mammary carcinoma through the combination of standard ICT treatment plus potent innate immune activating TLR5 agonists, 2) immune-related treatments elicited immune memory against tumor antigens in most long-term survivors, 3) systemic cytokine profiles implicated engagement of both innate and adaptive immunity in response to treatment, and 4) the data supports the idea that G-CSF may function as a bio-marker for positive response to treatment.

These support the approach of using a combination of these therapeutics to harnesses both innate and adaptive components of the immune system to elicit a lasting antitumor response. On the one hand, bacterial derived-flagellin has been shown to elicit a targeted antitumor response by binding to TLR5 on the tumor surface, initiating a cascade of signals that produce a pro-inflammatory response via activation of the transcription factor NF-kB (Flentie et al., 2018). The results provided herein show that in vitro CBLB502, a potent activator of the NF-kB signaling pathways, was sufficient to elicit a TLR5-mediated immunogenic cytokine response in tumor cells. Given that deficiencies in antigen presentation underlie many mechanisms of resistance against immune checkpoint therapies, it is possible to hypothesize that a potent activator of innate immunity may modulate tumor homeostasis, shifting the tumor microenvironment state from immune suppression to immune activation. Several lines of evidence support this model. First, only treatment with either flagella or CBLB502 in combination with ICT increased survival in mice bearing highly ICT-refractory 4T1 tumors, whereas monotherapies of flagellin, CBLB502, or ICT did not show significant curative effects. Second, the peripheral blood cytokine profiles of mice that responded to treatment and showed complete tumor regression reflected a concerted antitumor response, with IL-13, IL-9, CCL3 and CXCL1 showing higher levels in mice that responded to treatment. Third, nearly all survivor mice that were re-challenged with the same tumor rejected it, implying an adaptive response against tumor cells. Fourth, the peripheral cytokine profiles of those mice that were re-challenged and rejected the tumor aligned with a strong adaptive immune-activating response. While it has been shown that mice bearing tumor cells lacking TLR5 fail to respond to treatment with flagellin (Rhee et al., 2008), another study suggests that antitumor effects are mediated by TLR5 agonists acting on immune cells (Geng et al., 2015). It remains to be studied whether flagella and CBLB502 act on the tumor cells or on immune cells to elicit a curative immune response in the context of combination treatment with ICT.

Finally, the remarkable increase of blood-borne G-CSF protein levels in tumor-bearing mice that either failed treatment or served as tumor-bearing untreated controls suggested that G-CSF levels may be explored as a potential biomarker. Conversely, low serum level of G-CSF in tumor-bearing mice that responded to treatment or developed long-term tumor immunity suggested utility as a predictive marker for treatment response. Moreover, these results further raise caution against the use of G-CSF to prevent neutropenia in cancer patients. Although a recent meta-analysis study showed some benefit of supportive G-CSF therapy in overall survival of patients receiving chemotherapy, data also show an increased risk of developing secondary malignancies (Lyman et al., 2018). G-CSF therapy in the context of ICT remain to be explored.

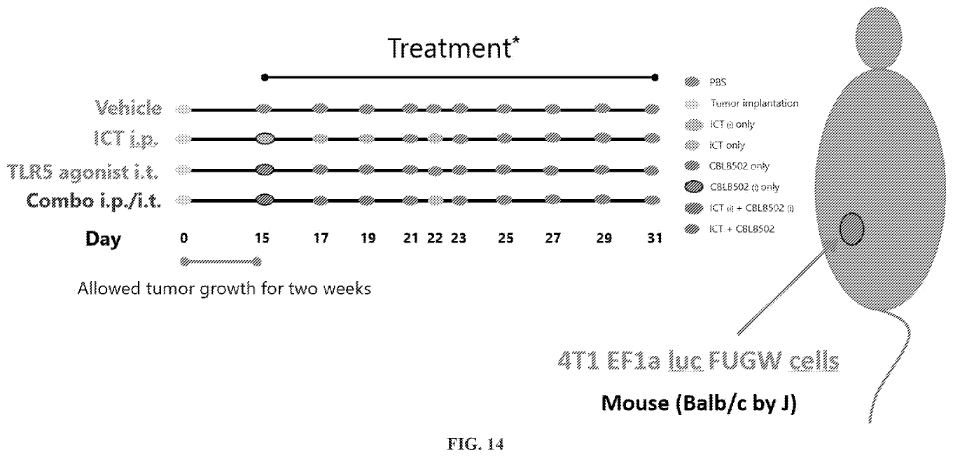

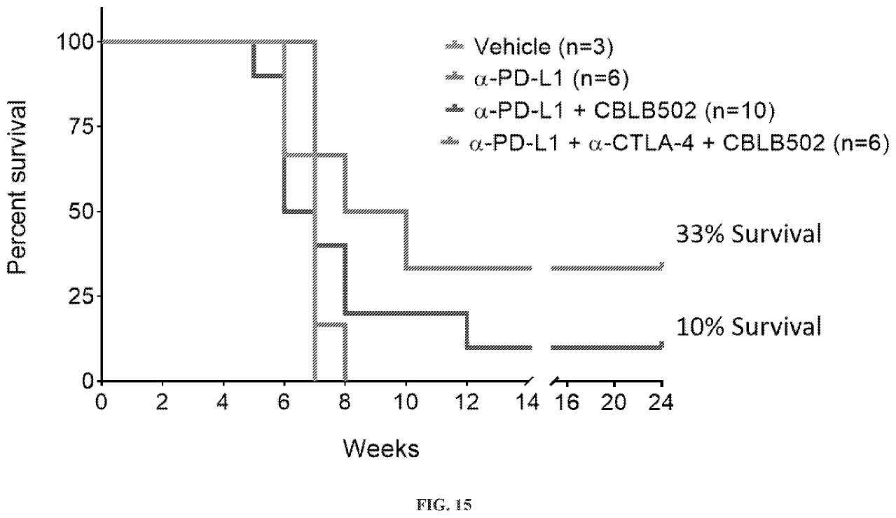

Additional studies demonstrated that CBLB502 in combination with an anti-PDL1 therapy improved responses in an immunotherapy-resistant model of triple negative breast cancer. Experiments were performed as follows: 10,000 4T1 EF1a luc FUGW-FL were implanted via Orthotopic mammary fat pad injection into 6 weeks old female Balb/c mice. After two weeks of tumor growth mice were treated for two weeks with: 1. Vehicle (n=3), 2. α-PD-L1 (n=6), 3. α-PD-L1+CBLB502 (n=10), 4. α-PD-L1+α-CTLA-4+CBLB502 (n=6). Dosing for each treatment was as follows: CBLB502—Initial (i) 1 μg/mouse follow by 200 ng/mouse every other day; Immune Checkpoint Therapy—Initial dose (i) of 200 μg/mouse per Ab (α-PD-L1 alone, or PD-L1 and α-CTLA-4) followed by 100 μg/mouse on days 17, 19 and 22 post initial dose. Antibodies InVivoMAb anti-mouse PD-L1 (B7-H1) (Cat. #BE0101, Clone: 10F.9G2), InVivoPlus anti-mouse CTLA-4 (CD152) (Cat. #BP0164, Clone: 9D9), and InVivoPlus anti-mouse PD-1 (CD279) (Cat. #BP0146, Clone: RMP1-14), all from BioXCell (Lebanon, N.H.) were used. The timing of the experiment, including tumor implantation and subsequent administration of therapeutic agents or vehicle (control group) are shown in FIG. 14. Results are shown in FIG. 15.

As shown in FIG. 15, similar to the results observed with the anti-PD1 therapy, CBLB502 enhanced survival for both: (i) when administered in combination with anti-PDL1 (10% long term survivors), and also (ii) further improved survival when administered in combination with both anti-PD-L1 and anti-CTLA4 treatment (33% long term survivors), and these results were observed in the strongly immunotherapy resistant model of triple negative breast cancer, 4T1. These results support the idea that administering CBLB502 in combination with a PD1 or PD-L1 therapy (e.g., an anti-PD1 antibody, an anti-PD-L1 antibody, etc.) can improve outcomes across the entire PD1 therapy axis.

In conclusion, these success of immune checkpoint therapy in eliciting long lasting curative responses against various types of cancers in subsets of patients make worthwhile efforts to expand the number of patients that respond to this type of treatment. The results provided herein support innate immune activators of TLR5, such as flagellin and CBLB502, in combination with immune checkpoint therapies may be used to treat cancers in vivo, and may beneficial for the treatment of previously unresponsive patients.

### Example 2

### Materials and Methods

Reagents

Salmonella typhimurium flagellin (FLA-ST) was purchased from Invivogen. CBLB502 was a gift from Cleveland Biolabs, Inc. Monoclonal antibodies 9D9 (anti-CTLA-4) and RPM1-14 (anti-PD-1) were purchased from BioX Cell and maintained in 6.5 mg/mL and 6.7 mg/mL stocks, respectively, and stored at 4° C. before use. d-luciferin (d-Luc) (BioGold), the substrate for firefly luciferase, was maintained in a 30 mg/mL solution of phosphate-buffered saline (PBS). Matrigel was obtain from Corning and maintain at −20° C.

Creation of 4T1 κB₅:IκBα-FLuc-Expressing Cell Line for In Vitro Study

4T1 mammary carcinoma cells (ATCC) at 95% confluency were co-transfected with 10 μg of pκB₅:IκBα-FLuc (Moss et al., 2012) and 3 μg of pIRES-puro plasmid DNA using Fugene 6 (Roche) in 10 cm dishes (BD Bioscience). After 24 hours, the media was replaced with fresh RPMI supplemented with 10% heat-inactivated FBS media. 24 hours later, cells were split at multiple dilutions into media containing 0.5 μg/ml puromycin to select for stable transformants. After two weeks, isolated cell colonies were imaged to confirm reporter gene expression and bioluminescent colonies were harvested and expanded. Reporter cells were continuously cultured in the presence of 0.5 μg/ml puromycin to maintain expression of the reporter plasmid.

In Vivo Study Cell Lines

4T1 mammary carcinoma cells were stably transfected with the EF1α:FLuc plasmid producing a constitutive florescent and bioluminescent dual imaging reporter cell line (4T1 FUGW-FL) (Luker et al. 2004). Cells were cultured according to ATCC protocols and kept under selection with 0.5 μg/ml puromycin. B16-F10 parental cells were cultured according to ATCC protocols (Fidler I J, 1975).

In Cellulo Analysis of NF-κB Signaling

4T1 κB₅:IκBα-FLuc reporter cells (7,000 cells) were added to a 96-well plate and incubated overnight at 37° C. One hour prior to imaging, cell media were aspirated and replaced with RPMI with L-Glutamate (4T1 cells) supplemented with 10% heat-inactivated FBS and 150 μg/ml d-luciferin (BioGold). Cells were imaged in an IVIS 100 imaging system, with images being acquired every 5 minutes for 4 hours, unless otherwise indicated. Cells were maintained in the imaging chamber by a heated stage (37° C.) and 5% CO₂ air flow. Acquisition parameters were: acquisition time, 60 sec; binning, 4-8; filter, open; f stop, 1; FOV, 12-23 cm. Stimuli included: TNFα (20 ng/ml) (R & D systems); flagellin (various concentrations ranging from 1 μg/mL to 0.1 ng/mL); CBLB502 (various concentrations ranging from 1 μg/mL to 0.1 ng/mL); and nuclease-free water (vector only control) added to triplicate wells. Bioluminescence photon flux data (photons/sec) represent the mean of triplicate wells for the indicated number of independent experiments, and were analyzed by region of interest (ROI) measurements with Living Image 3.2 (Caliper Life Sciences). Data were imported into Excel (Microsoft Corp.), averaged, and normalized to both initial (t=0) values (fold-initial) and vehicle-treated controls (fold-vehicle) for presentation in dynamic plots (Gross and Piwnica-Worms, 2005). The normalized results from repeated experiments were averaged for each time point, and the results graphed as normalized photon flux versus time, with the y-axis on a log 2 scale. Positive error bars present standard error of the mean for repeated experiments.

Mice

All animal procedures were approved by the Institutional Animal Care and Use Committee (IACUC) of the University of Texas M.D. Anderson Cancer Center, protocol 00001179-RN01. Female BALB/c mice (4 weeks old) were purchased from The Jackson Laboratory. Animals were allowed at least one week to acclimate to the animal facility before start of experiments. Age-matched control groups were used where indicated.

Mouse Endpoint Protocol

Mice that reached end point (moribund condition or having one tumor measurement in the sagittal or axial plane greater than 1.5 cm) were euthanized according to University of Texas M.D. Anderson Cancer Center IACUC euthanasia protocols.

Allograft Model of Human Breast Cancer

A mammary cell carcinoma allograft was established in each BALB/c mouse (5-6 week old mice) by orthotopic injection into the fourth mammary fat pad of approximately 10,000 4T1 FUGW-FL cells mixed with Matrigel at a 2:1 ratio. The total volume injected into each mouse was 30 μL.

Administration of Flagellin, CBLB502, and Immune Checkpoint Therapy for Allograph for Breast Cancer Carcinoma Model

Flagellin, CBLB502, 9D9 (anti-CTLA-4), and RPM1-14 (anti-PD-1) were suspended in filtered PBS; filtered PBS was used as a vehicle control. Two weeks post orthotopic injection of 4T1 FUGW-FL cells into the mammary fat pad, each mouse was randomly sorted into groups receiving vehicle control (n=40, total), or treatment with flagellin only (n=22), CBLB502 only (n=30), ICT only (9D9 plus RPM1-1) (n=37), flagellin combined with ICT or CBLB502 combined with ICT (n=52). Flagellin or CBLB502 was administered every two days for two weeks. On the first day of treatment, 10 μg of flagellin solution in 50 μL (PBS), or 10 μg (high dose) or 1 μg (low dose) of CBLB502 solution in 50 μL (PBS) were administered as an intra-tumoral or intraperitoneal injection into designated animals. For each subsequent treatment, 2 μg of flagellin solution in 50 μL (PBS) or 2 μg (high dose) or 0.5 μg (low dose) of CBLB502 solution in 50 μL (PBS) was used. ICT was administered on days 1, 3, 5, and 8 of treatment. On the first day, mice receiving ICT treatment were injected intraperitoneally with 200 μg in 100 μL of both 9D9 and RPM1-14 (200 μL total per mouse). On subsequent days, each mouse was injected with 100 μg in 100 μL of each antibody. On days when both flagellin and ICT were administered, vehicle control mice were given two intraperitoneal injections of 100 μL PBS and one intra-tumoral injection of 50 μL PBS. On days when only flagellin was administered, vehicle mice received only 50 μL PBS intra-tumoral injection, and on the day that only ICT was administered, they received only two 100 μL intraperitoneal injections of PBS.

In Vivo Bioluminescence Imaging

The mice were imaged using the PerkinElmer IVIS Spectrum Imaging System weekly beginning one week after orthotopic injection of 4T1 FUGW-FL cells into the mammary fat pad. The mice were weighed at the beginning of each imaging session, and 165 μg d-luciferin (prepared at 30 mg/mL in PBS) was injected intraperitoneally per gram of mouse. Mice were imaged ten minutes after injection with d-luciferin (Gross and Piwnica-Worms, 2005).

Allograft Model of Melanoma Tumor

A melanoma tumor was established in each C57BL/6J mouse (Jackson Laboratory, 6-9 week old mice) by subcutaneous injection into the right dorsal flank of approximately 12,000 B16-F10 cells. The total volume injected into each mouse was 50 μL of cells resuspend in RPMI 1640 with L-glutamine media (Millipore Sigma).

Administration of CBLB502 and Immune Checkpoint Therapy Treatments for Melanoma Tumor Model

Three days post subcutaneous injection of B16-F10 cells into the posterior right flank, each mouse was randomly sorted into a group receiving treatment with vehicle control, CBLB502 only, ICT only (9D9 plus RPM1-1), or CBLB502 combined with ICT. CBLB502, 9D9, and RPM1-14 were suspended in filtered PBS, and filtered PBS was used as a vehicle control. CBLB502 was administered every two days for two weeks. On the first day of treatment, 1 μg in 50 μL of CBLB502 was administered as an intra-tumoral injection into designated animals. For each subsequent treatment, 500 ng in 50 μL of CBLB502 was used. ICT was administered on days 1, 3, 5, and 8 of treatment. On the first day, mice receiving ICT treatment were injected intraperitoneally with 200 μg in 100 μL of both 9D9 and RPM1-14 (200 μL total per mouse). On subsequent treatment days, each mouse was injected with 100 μg in 100 μL of each antibody. On days when both CBLB502 and ICT were administered, vehicle control mice were given an intraperitoneal injections of 200 μL PBS and one intra-tumoral injection of 50 μL PBS. On days when only CBLB502 was administered, vehicle mice received only 50 μL PBS intra-tumoral injection, and on the day that only ICT was administered, they received only a 200 μL intraperitoneal injections of PBS.

Caliper Measurement of Tumor Volume

Tumor volume was determined by measuring length (1, longest measurement) and width (w) for each tumor at least once a week by caliper, using the standard triangular prism formula for volume: V=(l×w²)/2.

In Vitro Cytokine Profile

4T1 FUGW-FL florescent and bioluminescent dual reporter cells were plated in 100 mm tissue culture plates (BD) (750,000 cells per plate) and incubated overnight at 37° C. with RPMI supplemented with 10% heat-inactivated FBS. On day two, cell media were aspirated and replaced with RPMI without 10% heat-inactivated FBS. On day three, cultures were treated with either CBLB502 at 1 μg/ml or PBS as vector control in triplicates. On day four, media was collected into 15 ml tubes, centrifuge at 2,000 rpm at 4° C. for 10 minutes. The supernatant was assayed using Mouse Cytokine Antibody Array C series 1000 (RayBiotech).

Serum Collection

Serum from mice was obtained by mandibular bleeding once a week for the duration of the experiment. Samples were allowed to clot at room temperature for 1 hour, centrifuge at 2,000 g for 10 minutes at room temperature and sera (upper phase) was collected into Eppendorf tubes. All serum samples were stored at −80° C.

Luminex Multiplex Quantitative Analysis

Serum samples were analyzed at the Antibody-Based Proteomics Core at Baylor College of Medicine, Houston, Tex. The core used the Milliplex Mouse 32-Plex Cytokine Panel (Millipore), which included the following cytokines: G-CSF, GM-CSF, IFN-7, IL-1α, IL-10, IL-2, IL-3, IL-4, IL-5, IL-6, IL-7, IL-9, IL-10, IL-12 (p40), IL-12 (p70), IL-13, IL-15, IL-17, CXCL10, CXCL-1-like, LIF, CXCL5, CCL2, M-CSF, CXCL9, CCL3, CCL4, CXCL2, CCL5, TNF-α, VEGF and appropriate controls and calibration standards.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,399,363
U.S. Pat. No. 5,466,468
U.S. Pat. No. 5,543,158
U.S. Pat. No. 5,641,515
U.S. Pat. No. 6,613,308
U.S. Pat. No. 10,265,390
U.S. Pub. No. 2012/0208871
Aliper et al., Cancer Med; 3:737-46, 2014.
al-Ramadi et al., Clin Immunol; 130:89-97, 2009.
Aslakson and Miller, Cancer Res; 52:1399-405, 1992.
Brackett et al., Proc Natl Acad Sci USA; 113:E874-83, 2016.
Burdelya et al., Proc Natl Acad Sci USA; 110:E1857-66, 2013.
Burdelya et al., Science; 320:226-30, 2008.
Cai et al., Cancer Res; 71:2466-75, 2011.
Dexter et al., Cancer Res; 38:3174-81, 1978.
Entolimod in treating patients with locally advanced or metastatic solid tumors that cannot be removed by surgery. https://ClinicalTrials.gov/show/NCT01527136.
Fidler I J, Cancer Res; 35:218-24, 1975
Flentie et al., Cancer Discov; 2:624-37, 2012.
Flentie et al., Mol Cancer Res; 16:986-99, 2018.
Forbes, Nat Rev Cancer; 10:785-94, 2010.
Frahm et al., MBio.; 6, 2015.
Fujiwara et al., Osaka City Med J; 57:79-84, 2011.
Fukutomi et al., Surg Today; 42:288-91, 2012.
Galli et al., J Immunol; 184:6658-69, 2010.
Ganai et al., Br J Cancer; 101:1683-91, 2009.
Garaude et al., Sci Transl Med; 4:120ra16, 2012.
Geng et al., Cancer Res; 75:1959-71, 2015.
Goldberg and Sondel, Semin Oncol; 42:562-72, 2015.
Gross and Piwnica-Worms, Nat Methods; 2:607-14, 2005.
Hajam et al., Experimental & Molecular Medicine, 49:373, 2017.
Hayashi et al., Int J Mol Med; 10:3-10, 2002.
Hayashi et al., Nature; 410:1099-103, 2001.
Hossain et al., PLoS One; 9: 2014.
Kawasaki and Kawai, Front Immunol; 5:461, 2014.
Larkin, et al. N. Engl. J. Med., 373, 23-34, 2015.
Lechner et al., J Immunother; 36:477-89, 2013.
Leigh et al., PLoS One; 9: 2014.
Liakou, et al., Proc. Natl Acad. Sci. 105, 14987-14992, 2008.
Lois et al., Science; 295:868-72, 2002.
Lu, Frontiers Immunology; 5, 2014.
Luker et al. 2004.
Lyman et al., Ann Oncol; 29:1903-10, 2018.
Matsuda et al., Surg Today; 39:990-3, 2009.
Menendez et al., PLoS Genet; 7, 2011.
Moss et al., J Biol Chem; 283:8687-98, 2008.
Moss et al., J Biol Chem; 287:31359-70, 2012.
Multiple myeloma trial of orally administered Salmonella based Survivin vaccine. https://ClinicalTrials.gov/show/NCT03762291.
Nguyen et al., Vaccine; 31:3879-87, 2013.
Pawelek et al., Cancer Res; 57:4537-44, 1997.
Pawelek et al., Lancet Oncol; 4:548-56, 2003.
Rakoff-Nahoum and Medzhitov, Nature Reviews Cancer; 9:57-63, 2009.
Remington: The Science and Practice of Pharmacy, 21^{st} Ed., Lippincott Williams and Wilkins,
2005.
Remington's Pharmaceutical Sciences” 15th Ed., pages 1035-1038 and 1570-1580.
Remington's Pharmaceutical Sciences, 18th Ed., Mack Printing Company, pp. 1289-1329,
1990.
Remington: The Science and Practice of Pharmacy, 21^{st} Ed., Lippincott Williams and Wilkins,
2005.
Rhee et al., Gastroenterology; 135:518-28, 2008.
Schachter et al., Lancet., October 21; 390(10105):1853-1862, 2017.
Sfondrini et al., J Immunol; 176:6624-30, 2006.
Simone et al., Open Microbiol J; 3:1-8, 2009.
Smith et al., Cancer Res; 64:8604-12, 2004.
Song et al., Nature Communications; 9:2237, 2018.
Stathopoulos et al., Oncol Rep; 25:1541-4, 2011.
Xu et al., Ann Transl Med., August; 4(16): 301, 2016.
Yang et al., Oncotarget; 7:2936-50, 2016.
Yu et al., Sci Rep; 2:436, 2012.
Zheng et al., Sci Transl Med; 9:eaak9537, 2017.
Zhou et al., Asian Pac J Cancer Prev; 13:2807-12, 2012.

What is claimed is:

1. A method of treating a cancer in a mammalian subject, comprising administering to the subject a therapeutically effective amount of:
(i) a TLR5 agonist; and
(ii) an immune checkpoint therapy (ICT).

2. The method of claim 1, wherein the TLR5 agonist is flagellin or a flagellin derivative.

3. The method of claim 2, wherein the TLR5 agonist is flagellin, CBLB502, or a CBLB502 derivative.

4. The method of claim 3, wherein the immune checkpoint therapy comprises an anti-PD1 antibody, an anti-PD-L1 antibody, an anti-CTLA4 antibody; an anti-LAG3 antibody, an anti-TIM-3 antibody, an anti-VISTA antibody, an anti-TIGIT antibody, an anti-KIR antibody, an anti-CD47 antibody, an anti-B7-H3 antibody, an anti-B7-H4 antibody, an ICOS agonist, an OX40 agonist, and/or an IDO inhibitor.

5. The method of claim 4, wherein the immune checkpoint therapy comprises: (a) an anti-PD1 antibody or an anti-PD-L1 antibody; and (b) an anti-CTLA4 antibody.

6. The method of any one of claims 2-5, wherein the anti-PD1 antibody is Pembrolizumab, Nivolumab, REGN2810, BMS-936558, SHR1210, IBI308, PDR001, BGB-A317, BCD-100, or JS001.

7. The method of any one of claims 2-6, wherein the anti-PDL1 antibody is Avelumab, Atezolizumab, Durvalumab, KN035, MPDL3280A, MEDI4736, or BMS-936559.

8. The method of any claims 2-7, wherein the anti-CTLA4 antibody is Ipilimumab or Tremelimumab.

9. The method of any one of claims 3-8, wherein the CBLB502 is administered to the subject.

10. The method of claim 9, wherein about 5 μg/mL to 150 μg/mL of CBLB502 is administered to the subject.

11. The method of any one of claims 2-10, wherein the administration is intratumoral, peritumoral, intravenous, parenteral, subcutaneous, or intrathecal.

12. The method of claim 11, wherein the administration is intratumoral or peritumoral.

13. The method of claim 3, wherein CBLB502, an anti-PD1 antibody, and an anti-CTLA4 antibody are administered to the subject.

14. The method of claim 13, wherein the administration is intratumoral, peritumoral, intravenous, parenteral, subcutaneous, or intrathecal.

15. The method of claim 14, wherein the administration is intratumoral or peritumoral.

16. The method of any of claims 2-15, wherein the cancer is an ICT-refractory cancer or an ICT-refractory solid tumor.

17. The method of any of claims 2-16, wherein the cancer is a melanoma, a breast cancer, a lung cancer, a prostate cancer, a pancreatic cancer, a head and neck cancer, a liver cancer, an ovarian cancer, a nonpalpable cancer, or a lymphoma.

18. The method of claim 17, wherein the cancer is a melanoma or a breast cancer.

19. The method of claim 18, wherein the breast cancer is triple negative breast cancer.

20. The method of any one of claims 2-18, wherein the subject is a human, dog, cat, horse, or cow.

21. The method of claim 20, wherein the subject is a human.

22. A pharmaceutical composition comprising CBLB502, and an immune checkpoint therapy (ICT), wherein the pharmaceutical composition is formulated for injection, intratumoral administration, or peritumoral administration.

23. The composition of claim 22, wherein the immune checkpoint therapy is an anti-PD1 antibody, an anti-PD-L1 antibody, or an anti-CTLA4 antibody.

24. The composition of claim 23, wherein the pharmaceutical composition comprises both an anti-PD1 antibody and an anti-CTLA4 antibody.

25. The composition of claim 23, wherein the pharmaceutical composition comprises both an anti-PD-L1 antibody and an anti-CTLA4 antibody.

26. The composition of claim 23, wherein the pharmaceutical composition comprises an anti-PD1 antibody, an anti-PD1 antibody, and an anti-CTLA4 antibody.

27. The composition of any one of claims 22-26, wherein the anti-PD1 antibody is Pembrolizumab, Nivolumab, REGN2810, BMS-936558, SHR1210, IBI308, PDR001, BGB-A317, BCD-100, or JS001.

28. The composition of any one of claims 22-27, wherein the anti-PDL1 antibody is Avelumab, Atezolizumab, Durvalumab, KN035, MPDL3280A, MEDI4736, or BMS-936559.

29. The composition of any claims 22-28, wherein the anti-CTLA4 antibody is Ipilimumab or Tremelimumab.
